(19) United States
(12) Patent Application Publication
(10) Pub. No.: US 2025/0115557 A1
(43) Pub. Date: Apr. 10, 2025

(54) TOTAL SYNTHESES OF SELENONEINE, ISO-SELENONEINE, AND ISOMERS

(71) Applicant: UNIVERSITÉ LAVAL, Québec (CA)

(72) Inventors: Adel ACHOUBA, Québec (CA); Pierre AYOTTE, Québec (CA)

(73) Assignee: UNIVERSITÉ LAVAL, Québec (CA)

(21) Appl. No.: 18/730,990

(22) PCT Filed: Feb. 16, 2023
(86) PCT No.: PCT/CA2023/050197

§ 371 Date: Jul. 22, 2024

Related U.S. Application Data
(60) Provisional application No. 63/268,089, filed on Feb. 16, 2022.

(51) Int. Cl. C07D 233/66

(52) U.S. Cl. CPC C07D 233/66

(57) ABSTRACT

A process for the total synthesis of selenoneine, iso-selenoneine, and isomers thereof is disclosed herein. Regarding the synthesis of selenoneine, the process comprises reacting an alkylated L-histidine methyl ester with a stable, acid-labile alkylating agent followed by reaction with elemental selenium under mildly basic conditions; and an acidic hydrolysis step. Regarding the synthesis of iso-selenoneine, the process comprises reacting hercynine with an electrophilic R—Se—X species, wherein the R—Se—X species is obtained by reaction of an alkyl diselenide with X₂, and wherein X is F, Cl, Br or I; and a deprotection reaction.

### CROSS REFERENCE TO RELATED APPLICATIONS

This application claims the benefit of U.S. Provisional Application 63/268,089, filed Feb. 16, 2022. The contents of the referenced application are incorporated into the present application by reference.

### BACKGROUND

### 1. Field

This disclosure relates to the field of chemistry. More specifically, but not exclusively, the present disclosure broadly relates to the total synthesis of selenoneine, iso-selenoneine, and poly-seleno-hercynine. Yet more specifically, but not exclusively, the present disclosure broadly relates to the total synthesis of L-(+)-selenoneine.

### 2. Related Art

Selenium (Se) is an essential trace element which exists in both organic and inorganic forms in living organisms. Se is incorporated as selenocysteine (SeCys), the 21^{st} amino acid, in selenoproteins. A total of 25 selenoproteins have been identified that contain a SeCys residue in their active site and which are noted to have several biological functions, notably protection from oxidative stress.^{[1,2]} Among the other organic forms, a novel organoselenium compound, selenoneine, stands out as being the first naturally occurring compound bearing a selone group in an imidazole moiety (FIG. 1; compound 1a).

Selenoneine was first isolated from the blood of bluefin tuna, Thunnus orientalis, in 2010.^{[3]} The methylated metabolite, Se-methylselenoneine, was subsequently identified in human urine.^{[4]} Although not synthesized by humans, selenoneine was found to be the major selenocompound in red blood cells (RBCs) of a fish-eating population on remote Japanese islands.^{[5]} More recently, it was found that selenoneine was the major Se species in RBCs of Inuit adults from Nunavik, and that beluga mattaaq (skin with the underlying fat layer), a highly praised delicacy, was the main source of selenoneine in the Inuit diet.^{[6,7]}

Selenoneine was found to exhibit strong antioxidant properties,^{[3]} methylmercury detoxifying capacity,^{[8]} and inhibitory activity against the angiotensin-converting enzyme (ACE).^{[9]} Furthermore, selenoneine is the analog of the well-known natural antioxidant ergothioneine (FIG. 1; compound 1b). The presence of Se in place of sulfur (S) enhances the attributes of this molecule, due to the differences in chemical properties between the two chalcogens. Being a heavier element relative to S, Se is more polarizable, which makes it more nucleophilic and more electrophilic than S.^{[10]} Se also possesses a greater tolerance for hypervalency, thereby enhancing its electrophilic properties.^{[11]} Moreover, selenols are more acidic than thiols, by 3 to 4 pKₐ units, and also possess a greater reducing potential.^{[12,13]}

As of now, only one chemical synthesis of selenoneine was published, and besides not being conducive to large scale synthesis, leads to a racemic mixture thereof.^{[14]} To more fully comprehend and study the chemical properties and the potential health benefits of selenoneine, an improved synthetic route which allows for easier access to L-(+)-selenoneine is of commercial interest.

### SUMMARY

The present disclosure broadly relates to the total synthesis of selenoneine, iso-selenoneine, and isomers thereof. More specifically, but not exclusively, the present disclosure broadly relates to the stereoselective total synthesis of both enantiomeric forms of selenoneine. More specifically, but not exclusively, the present disclosure broadly relates to the stereoselective total synthesis of L-(+)-selenoneine. The present disclosure also relates to the synthesis of iso-selenoneine and poly-seleno-hercynine.

In an aspect, the present disclosure relates to a process for preparing selenoneine, the process comprising a selenation step using a stable acid-labile alkylating agent, and an acidic hydrolysis step. In an embodiment of the present disclosure, the process comprises reacting an alkylated histidine methyl ester with a stable, acid-labile alkylating agent followed by reaction with elemental selenium under mildly basic conditions. In an embodiment of the present disclosure, the process further comprises an acidic hydrolysis step.

In an aspect, the present disclosure relates to a process for preparing L-(+)-selenoneine, the process comprising a selenation step using a stable acid-labile alkylating agent, and an acidic hydrolysis step. In an embodiment of the present disclosure, the process comprises reacting an alkylated L-histidine methyl ester with a stable, acid-labile alkylating agent followed by reaction with elemental selenium under mildly basic conditions. In an embodiment of the present disclosure, the process further comprises an acidic hydrolysis step.

In an aspect, the present disclosure relates to a process for preparing iso-selenoneine, the process comprising reacting hercynine with an electrophilic R—Se—X species, wherein X is F, Cl, Br or I. In an embodiment of the present disclosure, the electrophilic R—Se—X species is obtained by reaction of an alkyl diselenide with I₂. In an embodiment of the present disclosure, the process further comprises a β-elimination reaction. In yet a further embodiment of the present disclosure, the alkyl group is a substituted alkyl group, wherein the substituent undergoes a β-elimination reaction under suitable reaction conditions. In yet a further embodiment of the present disclosure, the substituent is a cyano group.

In an aspect, the present disclosure relates to a process for preparing L-(+)-iso-selenoneine, the process comprising reacting L-hercynine with an electrophilic R—Se—X species, wherein X is F, Cl, Br or I. In an embodiment of the present disclosure, the electrophilic R—Se—X species is obtained by reaction of an alkyl diselenide with I₂. In an embodiment of the present disclosure, the process further comprises a β-elimination reaction. In yet a further embodiment of the present disclosure, the alkyl group is a substituted alkyl group, wherein the substituent undergoes a β-elimination reaction under suitable reaction conditions. In yet a further embodiment of the present disclosure, the substituent is a cyano group.

In an aspect, the present disclosure relates to a process for preparing poly-seleno-hercynine.

In an aspect, the present disclosure relates to a method of preparing a selenium compound of formula (I):

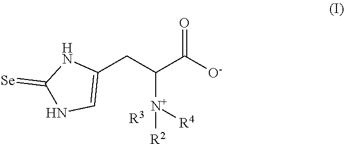

wherein R², R³ and R⁴ are each independently alkyl, the method comprising reacting a histidine ester under conditions sufficient to provide a dialkylated histidine ester; protecting the amino groups of the imidazole moiety of the dialkylated histidine ester and introducing a selone functional group at C2 of the imidazole moiety; deprotecting the amino groups of the imidazole moiety; protecting the imidazole N—H and selone functional groups; alkylating the tertiary amine and removing the protecting groups to provide the selenium compound of formula I.

In an aspect, the present disclosure relates to a method of preparing a selenium compound of formula (I):

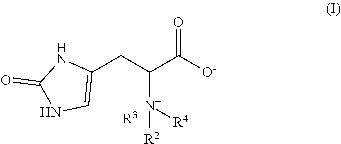

wherein R², R³ and R⁴ are each independently alkyl; the method comprising reacting a compound of formula 2′:

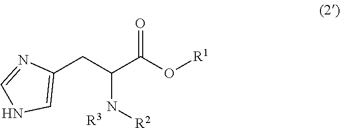

wherein R¹, R² and R³ are each independently alkyl, with an amine protecting agent followed by a selenation reaction to provide a compound of formula 3′:

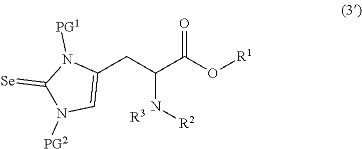

wherein R¹, R² and R³ are each independently alkyl and wherein PG¹ and PG² are amine protecting groups, and wherein PG¹ and PG² may be identical or different.

In an embodiment, the method of preparing a selenium compound of formula (I) further comprises removing the amino protecting groups from the compound of formula 3′, to provide a compound of formula 4′:

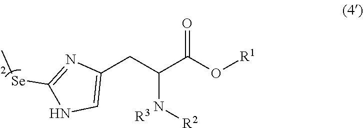

wherein R¹, R² and R³ are each independently alkyl.

In an embodiment, the method of preparing a selenium compound of formula (I) further comprises reacting the compound of 4′ with a protecting agent to provide a compound of formula 5′:

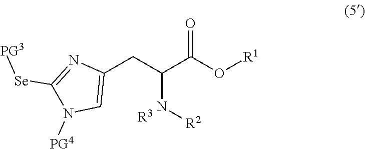

wherein R¹, R² and R³ are each independently alkyl and wherein PG³ and PG⁴ are amine protecting groups and a selenium protecting group respectively, wherein PG³ and PG⁴ may be identical or different.

In an embodiment, the method of preparing a selenium compound of formula (I) further comprises quaternization of the tertiary amine and removing the protecting groups from the compound of formula 5′, to provide the compound of formula I.

In an aspect, the present disclosure relates to a method of preparing a selenium compound of formula (II)

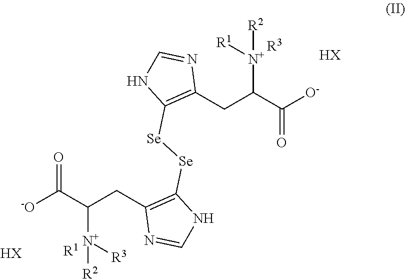

wherein R¹, R² and R³ are each independently alkyl and X is a halogen, the method comprising reacting a hercynine derivative under conditions sufficient for introducing a selenium at C5 of the imidazole moiety; and reacting the C5 seleno-substituted derivative to provide the selenium compound of formula II.

In an aspect, the present disclosure relates to a method of preparing a selenium compound of formula (II):

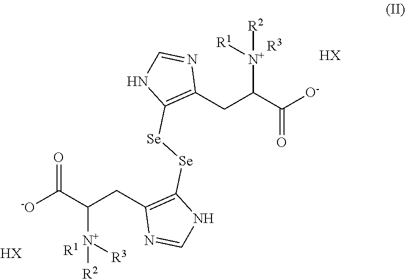

wherein R¹, R² and R³ are each independently alkyl and X is a halogen, the method comprising reacting a compound of formula 8′

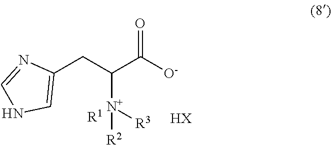

wherein R¹, R² and R³ are each independently alkyl and X is a halogen, with a selenation reagent to provide a compound of formula 9′

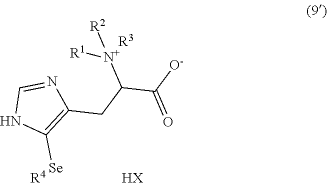

wherein R¹, R² and R³ are each independently alkyl; wherein R⁴ is a substituted alkyl group; and wherein X is a halogen.

In an embodiment, the method of preparing a selenium compound of formula (II) further comprises subjecting the compound of formula 9′ to a deprotection reaction to provide the compound of formula II.

In an aspect, the present disclosure relates to a method of preparing a selenium compound of formula (I):

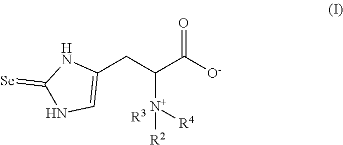

wherein R², R³ and R⁴ are each independently alkyl, the method comprising reacting a histidine ester under conditions sufficient to provide a dialkylated histidine ester; protecting the amino groups of the imidazole moiety of the dialkylated histidine ester and introducing a selone functional group at C2 of the imidazole moiety; protecting the selone functional group; alkylating the tertiary amine and removing the protecting groups to provide the selenium compound of formula I.

In an aspect, the present disclosure relates to a method of preparing a selenium compound of formula (I):

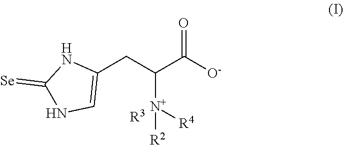

wherein R², R³ and R⁴ are each independently alkyl; the method comprising reacting a compound of formula 2′:

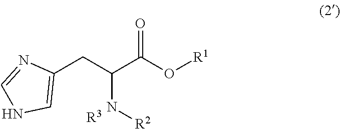

wherein R¹, R² and R³ are each independently alkyl, with an amine protecting agent followed by a selenation reaction to provide a compound of formula 3′:

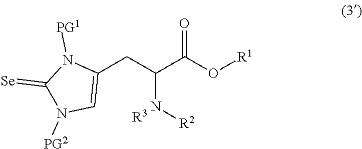

wherein R¹, R² and R³ are each independently alkyl and wherein PG¹ and PG² are amine protecting groups, and wherein PG¹ and PG² may be identical or different.

In an embodiment, the method of preparing a selenium compound of formula (I) further comprises reacting the compound of formula 3′ with a protecting agent to provide a compound of formula 13′:

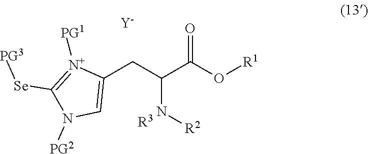

wherein R¹, R² and R³ are each independently alkyl, and wherein PG¹, PG² and PG³ are amine and selenium protecting groups respectively, wherein PG¹, PG² and PG³ may be identical or different.

In an embodiment, the method of preparing a selenium compound of formula (I) further comprises quaternization of the tertiary amine of the compound of formula 13′ to provide a compound of formula 14′:

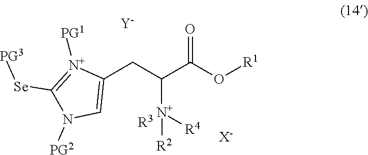

wherein R¹, R², R³ and R⁴ are each independently alkyl, and wherein PG¹, PG² and PG³ are amine and selenium protecting groups respectively, wherein PG¹, PG² and PG³ may be identical or different.

In an embodiment, the method of preparing a selenium compound of formula (I) further comprises removing the protecting groups from the compound of formula 14′, to provide the compound of formula I.

In an aspect, the present disclosure relates to a method for improving an antioxidant effect that involves selenium in a subject, the method comprising administering a composition containing an effective amount of a compound of formula I:

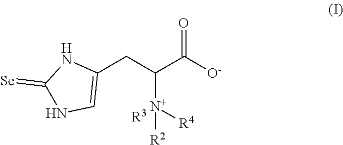

wherein R², R³ and R⁴ are each independently alkyl.

In an embodiment of the present disclosure, the subject is a human or an animal. In a further embodiment of the present disclosure, the composition is a drug, a functional food, a nutritional supplement, a food additive, an animal drug, a feed additive, or an antioxidant.

In an aspect, the present disclosure relates to a method for inhibiting oxidation in a cell or tissue, the method comprising administering a composition containing an effective amount of a compound of formula I:

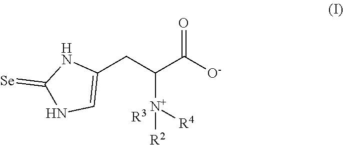

wherein R², R³ and R⁴ are each independently alkyl.

In an embodiment of the present disclosure, the composition is effective for inhibiting or treating cytotoxic effects caused by a reactive oxygen species and/or methylmercury chloride (MeHgCl). In an embodiment of the present disclosure, the reactive oxygen species may be a peroxide. In a further embodiment of the present disclosure, the peroxide may be t-butyl hydroperoxide (t-BuOOH).

In an aspect, the present disclosure relates to a radiolabelled selenoneine. In an embodiment, the present disclosure relates to a radiolabelled L-(+)-selenoneine. In a further embodiment of the present disclosure, the radiolabel may be one or more of D, T, Se⁷⁴, Se⁷⁶, Se⁷⁷, Se⁷⁸, and Se⁸⁰.

In some embodiments, one or more steps of the synthesis further comprises purifying the reaction in a purification step. In some embodiments, the purification method is chromatography. In some embodiments, the purification method is preparative thin-layer chromatography, column chromatography or high-performance liquid chromatography.

Also disclosed within the context of the present disclosure are embodiments 1 to 34. Embodiment 1 is a method of preparing a selenium compound of formula (I):

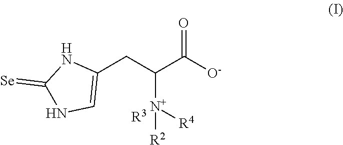

wherein R², R³ and R⁴ are each independently alkyl, the method comprising: reacting a histidine ester under conditions sufficient to provide a dialkylated histidine ester; protecting the amino groups of the imidazole moiety of the dialkylated histidine ester and introducing a selone functional group at C2 of the imidazole moiety; deprotecting the amino groups of the imidazole moiety; protecting the imidazole N—H and selone functional groups; alkylating the tertiary amine and removing the protecting groups to provide the selenium compound of formula I.

Embodiment 2 is a method of preparing a selenium containing compound of formula (I):

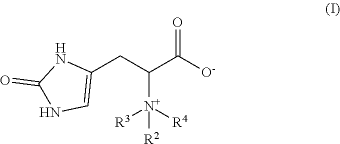

wherein R², R³ and R⁴ are each independently alkyl; the method comprising reacting a compound of formula 2′:

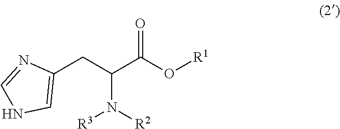

wherein R¹, R² and R³ are each independently alkyl, with an amine protecting agent followed by a selenation reaction to provide a compound of formula 3′:

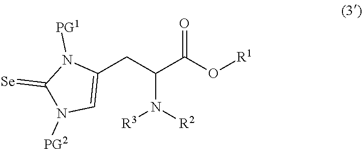

wherein R¹, R² and R³ are each independently alkyl and wherein PG¹ and PG² are amine protecting groups, and wherein PG¹ and PG² may be identical or different.

Embodiment 3 is the method of embodiment 2, further comprising removing the amino protecting groups from the compound of formula 3′, to provide a compound of formula 4′:

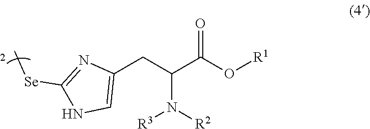

wherein R¹, R² and R³ are each independently alkyl.

Embodiment 4 is the method of embodiment 3, further comprising reacting the compound of formula 4′ with a protecting agent to provide a compound of formula 5′:

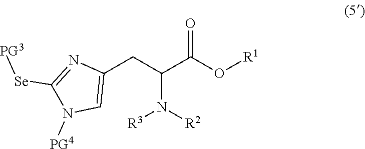

wherein R¹, R² and R³ are each independently alkyl and wherein PG³ and PG⁴ are an amine protecting group and a selenium protecting group respectively, wherein PG³ and PG⁴ may be identical or different.

Embodiment 5 is the method of embodiment 4, further comprising quaternization of the tertiary amine and removing the protecting groups from the compound of formula 5′, to provide the compound of formula I.

Embodiment 6 is the method of any one of embodiments 2 to 5, wherein the compound of formula 2′ is:

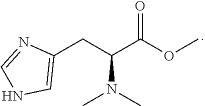

Embodiment 7 is the method of any one of embodiments 2 to 6, wherein the compound of formula 3′ is:

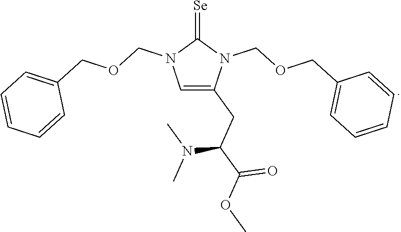 [SMILES: COC(=O)[C@H](Cc1cn(COCc2ccccc2)c(=[Se])n1COCc1ccccc1)N(C)C]

Embodiment 8 is the method of any one of embodiments 2 to 7, wherein the compound of formula 4′ is:

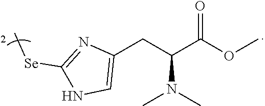

Embodiment 9 is the method of any one of embodiments 2 to 8, wherein the compound of formula 5′ is:

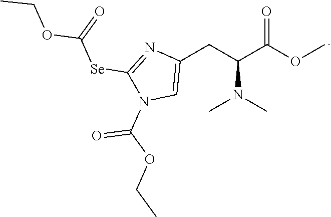 [SMILES: CCOC(=O)[Se]c1nc(C[C@@H](C(=O)OC)N(C)C)cn1C(=O)OCC]

Embodiment 10 is the method of any one of embodiments 2 to 9, wherein the compound of formula I is:

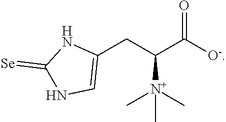

Embodiment 11 is a method of preparing a selenium compound of formula (II)

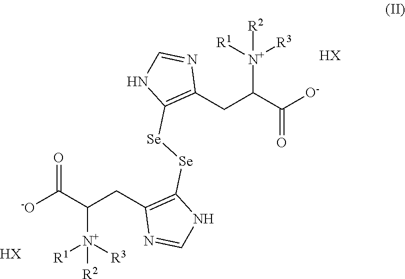

wherein R¹, R² and R³ are each independently alkyl and X is a halogen; the method comprising reacting a hercynine derivative under conditions sufficient for introducing a selenium at C5 of the imidazole moiety, and reacting the C5 seleno-substituted derivative to provide the selenium compound of formula II.

Embodiment 12 is a method of preparing a selenium containing compound of formula (II):

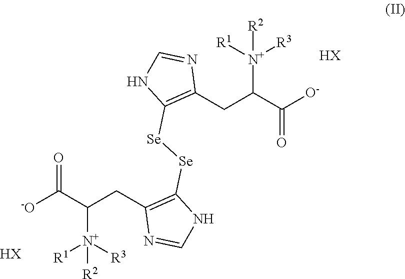

wherein R¹, R² and R³ are each independently alkyl and X is a halogen, the method comprising reacting a compound of formula 8′

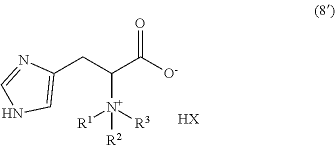

wherein R¹, R² and R³ are each independently alkyl and X is a halogen, with a selenation reagent to provide a compound of formula 9′

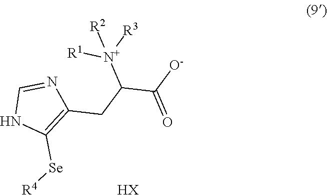

wherein R¹, R² and R³ are each independently alkyl; wherein R⁴ is a substituted alkyl group, wherein the substituent is a leaving group; and wherein X is a halogen.

Embodiment 13 is the method of embodiment 12, further comprising subjecting the compound of formula 9′, to a deprotection reaction to provide the compound of formula II.

Embodiment 14 is the method of embodiment 13, wherein the substitution reaction comprises a β-elimination reaction.

Embodiment 15 is the method of any one of embodiments 12 to 14, wherein the compound of formula 8′ is:

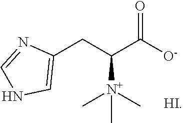

Embodiment 16 is the method of any one of embodiments 12 to 15, wherein the compound of formula 9′ is:

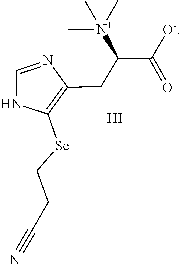

Embodiment 17 is the method of any one of embodiments 12 to 16, wherein the compound of formula II is:

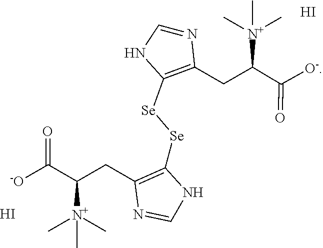

Embodiment 18 is a method of preparing a selenium compound of formula (I):

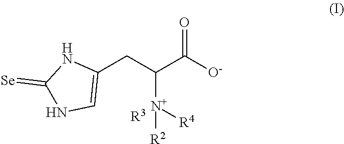

wherein R², R³ and R⁴ are each independently alkyl, the method comprising: reacting a histidine ester under conditions sufficient to provide a dialkylated histidine ester; protecting the amino groups of the imidazole moiety of the dialkylated histidine ester and introducing a selone functional group at C2 of the imidazole moiety; protecting the selone functional group; alkylating the tertiary amine and removing the protecting groups to provide the selenium compound of formula I.

Embodiment 19 is a method of preparing a selenium containing compound of formula (I):

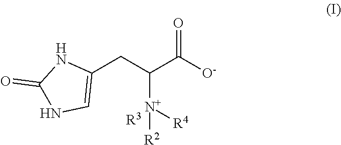

wherein R², R³ and R⁴ are each independently alkyl; the method comprising reacting a compound of formula 2′:

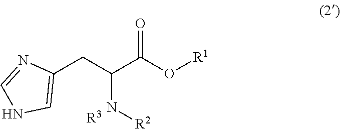

wherein R¹, R² and R³ are each independently alkyl, with an amine protecting agent followed by a selenation reaction to provide a compound of formula 3′:

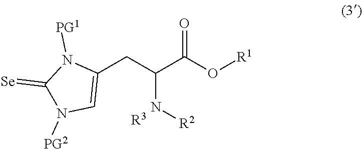

wherein R¹, R² and R³ are each independently alkyl and wherein PG¹ and PG² are amine protecting groups, and wherein PG¹ and PG² may be identical or different.

Embodiment 20 is the method of embodiment 19, further comprising reacting the compound of formula 3′ with a protecting agent to provide a compound of formula 13′:

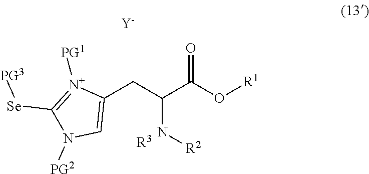

wherein R¹, R² and R³ are each independently alkyl, and wherein PG¹, PG² and PG³ are amine and selenium protecting groups respectively, wherein PG¹, PG² and PG³ may be identical or different.

Embodiment 21 is the method of embodiment 20, further comprising quaternization of the tertiary amine of the compound of formula 13′ to provide a compound of formula 14′:

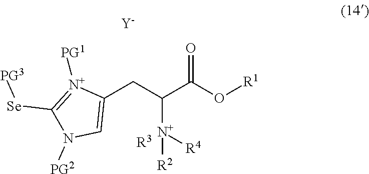

wherein R¹, R², R³ and R⁴ are each independently alkyl, and wherein PG¹, PG² and PG³ are amine and selenium protecting groups respectively, wherein PG¹, PG² and PG³ may be identical or different.

Embodiment 22 is the method of embodiment 21, further comprising removing the protecting groups from the compound of formula 14′, to provide the compound of formula I.

Embodiment 23 is the method of any one of embodiments 19 to 22, wherein the compound of formula 2′ is:

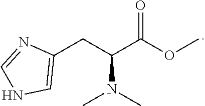

Embodiment 24 is the method of any one of embodiments 19 to 23, wherein the compound of formula 3′ is:

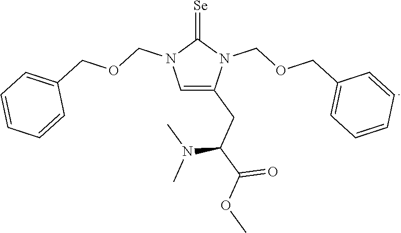 [SMILES: COC(=O)[C@H](Cc1cn(COCc2ccccc2)c(=[Se])n1COCc1ccccc1)N(C)C]

Embodiment 25 is the method of any one of claims 19 to 24, wherein the compound of formula 13′ is:

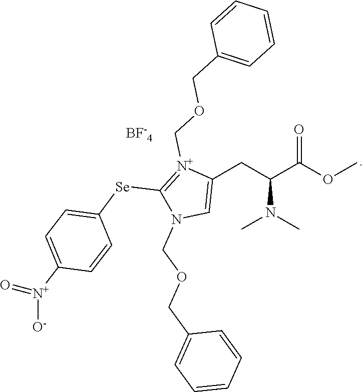 [SMILES: COC(=O)[C@H](Cc1cn(COCc2ccccc2)c([Se]c2ccc([N+](=O)[O-])cc2)[n+]1COCc1ccccc1)N(C)C ; "BF-4"]

Embodiment 26 is the method of any one of embodiments 19 to 25, wherein the compound of formula 14′ is:

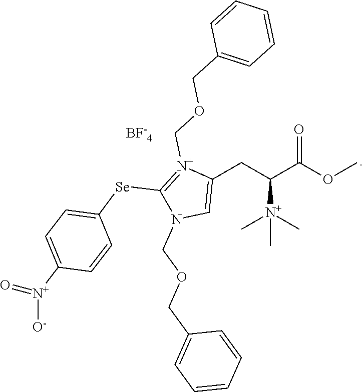 [SMILES: COC(=O)[C@H](Cc1cn(COCc2ccccc2)c([Se]c2ccc([N+](=O)[O-])cc2)[n+]1COCc1ccccc1)[N+](C)(C)C ; "BF-4"]

Embodiment 27 is the method of any one of embodiments 19 to 26, wherein the compound of formula I is:

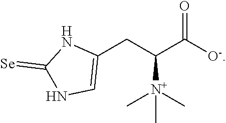

Embodiment 28 is a method for improving an antioxidant effect that involves selenium in a subject, the method comprising administering a composition containing an effective amount of a compound of formula I:

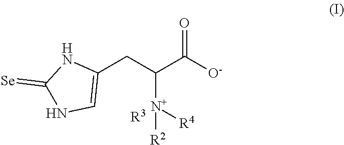

wherein R², R³ and R⁴ are each independently alkyl, and wherein the compound of formula I is obtained according to the method of any one of embodiments 1 to 10 or 19 to 28.

Embodiment 29 is the method of embodiment 28, wherein the subject is a human or an animal.

Embodiment 30 is the method of embodiment 28 or 29, wherein the composition is a drug, a functional food, a nutritional supplement, a food additive, an animal drug, a feed additive, or an antioxidant.

Embodiment 31 is a method for inhibiting oxidation in a cell or tissue, the method comprising administering a composition containing an effective amount of a compound of formula I:

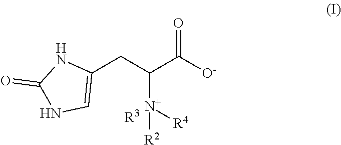

wherein R², R³ and R⁴ are each independently alkyl, and wherein the compound of formula I is obtained according to the method of any one of embodiments 1 to 10 or 19 to 28.

Embodiment 32 is the method of embodiment 31, wherein the composition is effective for inhibiting or treating cytotoxic effects caused by a reactive oxygen species and/or methylmercury chloride (MeHgCl).

Embodiment 33 is the method of embodiment 32, wherein the reactive oxygen species is a peroxide.

Embodiment 34 is the method of embodiment 33, wherein the peroxide is t-butyl hydroperoxide (t-BuOOH).

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The foregoing and other advantages and features of the present disclosure will become more apparent upon reading of the following non-restrictive detailed description of illustrative embodiments thereof, with reference to the accompanying drawings/figures. It should be understood, however, that the detailed description and the illustrative embodiments, while indicating specific embodiments of the disclosure, are given by way of illustration only, since various changes and modifications within the spirit and scope of the disclosure will become apparent to those skilled in the art from this description.

### BRIEF DESCRIPTION OF THE DRAWINGS/FIGURES

The following figures/drawings form part of the present specification and are included to further demonstrate certain aspects of the present specification. The present specification may be better understood by reference to one or more of these figures/drawings in combination with the detailed description. In the appended drawings/figures:

FIG. 1—Chemical structures of selenoneine (a), ergothioneine (b) and hercynine (c).

FIG. 2—Circular dichroism (CD) data of synthesized L-(+)-selenoneine 7 in accordance with an embodiment of the present disclosure.

FIG. 3—Illustration of the protective effect of selenoneine or ergothioneine against t-BuOOH induced oxidative stress in human erythroid K562 cells (830 μM; IC₅₀) in accordance with an embodiment of the present disclosure. Cell viability was assessed by the trypan blue exclusion test.

FIG. 4—Illustration of the protective effect of selenoneine or ergothioneine against MeHgCl induced oxidative stress in human erythroid K562 cells (5 μM; IC₅₀) in accordance with an embodiment of the present disclosure. Cell viability was assessed by the trypan blue exclusion test.

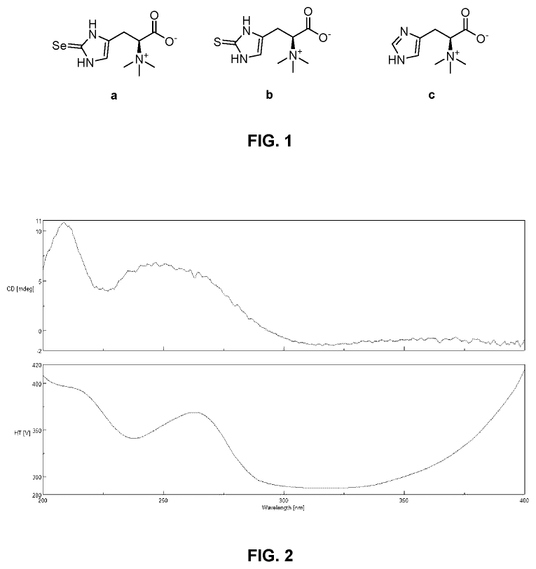

### DETAILED DESCRIPTION OF ILLUSTRATIVE EMBODIMENTS

The present disclosure relates to the total synthesis of selenoneine, iso-selenoneine, and isomers thereof. More specifically, but not exclusively, the present disclosure broadly relates to the stereoselective total synthesis of both enantiomeric forms of selenoneine. More specifically, but not exclusively, the present disclosure broadly relates to the stereoselective total synthesis of L-(+)-selenoneine. The present disclosure also relates to the synthesis of iso-selenoneine and poly-seleno-hercynine.

In an aspect, the present disclosure relates to a synthetic process for preparing L-(+)-selenoneine. The process advantageously comprises a selenation step using a stable acid-labile alkylating agent, and an acidic hydrolysis step. In an embodiment of the present disclosure, the synthetic process comprises reacting an alkylated L-histidine methyl ester with a stable, acid-labile alkylating agent followed by reaction with elemental selenium under mildly basic conditions. In an embodiment of the present disclosure, the process further comprises an acidic hydrolysis step. These and other aspects of the disclosure are described in greater detail below.

(±)-Selenoneine, L-(+)-selenoneine, iso-selenoneine and poly-seleno-hercynine can be synthesized according to the methods described, for example, in the Examples section below. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

(±)-Selenoneine contains an asymmetrically-substituted carbon atom and may be isolated in optically active or racemic form. In an embodiment of the present disclosure, L-(+)-selenoneine is advantageously obtained in optically active form. Thus, all chiral and racemic forms of a chemical formula are intended, unless the specific stereochemistry is specifically indicated. Compounds may occur as racemates, single enantiomers or, in the case of iso-selenoneine, also as diastereomers. In some embodiments, a single enantiomer is obtained. The chiral centers of the compounds of the present disclosure can have the S- or the R-configuration. The enantiomerically pure forms of the compounds of the present disclosure may rotate plane polarized light in a clockwise (+) or counterclockwise (−) direction.

In addition, atoms making up selenoneine, iso-selenoneine and poly-seleno-hercynine, are intended to include all isotopic forms of such atoms. Isotopes, as used herein, include those atoms having the same atomic number but different mass numbers. By way of general example and without limitation, isotopes of hydrogen include tritium and deuterium; isotopes of carbon include ¹³C and ¹⁴C; isotopes of selenium include Se⁷⁴, Se⁷⁶, Se⁷⁷, Se⁷⁸, Se⁷⁹ and Se⁸⁰; isotopes of nitrogen include N¹⁴ and N¹⁵; etc.

### Synthetic Methods

In some aspects the compounds of the present disclosure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

### Process Scale-Up

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein. The synthetic methods described herein may be used to produce preparative scale amounts of selenoneine, iso-selenoneine and poly-seleno-hercynine.

### Chemical Definitions

When used in the context of a chemical group: “hydrogen” means —H; “hydroxy” means —OH; “oxo” means ═O; “carbonyl” means —C(═O)—; “selenonyl” means —C(═Se)-“carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; “imino” means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfato” means —SO₃H, “sulfamido” means —S(O)₂NH₂, “sulfonyl” means —S(O)₂—; and “sulfinyl” means —S(O)—.

In the context of chemical formulas, the symbol “—.” means a single bond, “═” means a double bond, and “≡” means a triple bond. The symbol “ [symbol] ” represents an optional bond, which if present is either single or double. The symbol “ [symbol] ” represents a single bond or a double bond. Furthermore, it is noted that the covalent bond symbol “—.”, when connecting one or two stereogenic atoms, does not indicate any preferred stereochemistry. Instead, it covers all stereoisomers as well as mixtures thereof. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom.

As used herein, the term “alkyl” refers to straight-chain or branched-chain alkyl residues. This also applies if they carry substituents or occur as substituents on other residues, for example in alkoxy residues, alkoxycarbonyl residues or arylalkyl residues. Substituted alkyl residues are substituted in any suitable position. Examples of alkyl residues containing from 1 to 18 carbon atoms are methyl, ethyl, propyl, butyl, pentyl, hexyl, heptyl, octyl, nonyl, decyl, undecyl, dodecyl, tetradecyl, hexadecyl and octadecyl, the n-isomers of all these residues, isopropyl, isobutyl, isopentyl, neopentyl, isohexyl, isodecyl, 3-methylpentyl, 2,3,4-trimethylhexyl, sec-butyl, tert-butyl, or tert-pentyl. A specific group of alkyl residues is formed by the residues methyl, ethyl, n-propyl, isopropyl, n-butyl, isobutyl, sec-butyl and tert-butyl.

As used herein, the term “lower alkyl” refers to straight-chain or branched alkyl residues comprising 1 to 6 carbon atoms. This also applies if they carry substituents or occur as substituents on other residues, for example in alkoxy residues, alkoxycarbonyl residues or arylalkyl residues. Substituted alkyl residues can be substituted in any suitable position. Examples of lower alkyl residues containing from 1 to 6 carbon atoms are methyl, ethyl, propyl, isopropyl, butyl, isobutyl, tert-butyl, pentyl, isopentyl, neopentyl, and hexyl.

The terms “alkoxy” or “alkyloxy,” as used interchangeably herein, represent an alkyl group attached to the parent molecular group through an oxygen atom.

An “amino protecting group” is well understood in the art. An amino protecting group is a group which prevents the reactivity of the amino group during a reaction which modifies some other portion of the molecule and can be easily removed to generate the desired amino group. Amino protecting groups can be found at least in Greene and Wuts, 1999, which is incorporated herein by reference. Some non-limiting examples of amino protecting groups include alkoxymethyl groups such as MOM, MEM, SEM, BOM, M-BOM, BUM and NAPOM. In an embodiment of the present disclosure, the amino protecting groups are acid-labile and advantageously provide for the protection of both nitrogen atoms of the imidazole moiety. Moreover, the amino protecting groups, advantageously avoid racemization from occurring during their installment (“protection step”) as well as during their removal (“deprotection step”).

### EXAMPLES

The following examples are included to demonstrate preferred embodiments of the disclosure. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—General Synthetic Approach to Selenoneine Synthesis

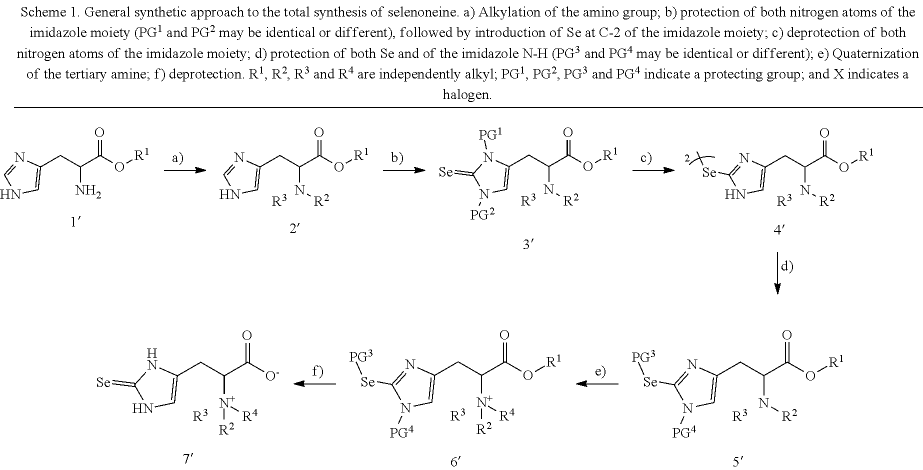

### Example 2—L-(+)-Selenoneine Synthesis

Two major hurdles are faced by chemists when attempting the synthesis of L-(+)-selenoneine. The first one being the hydrolysis-induced racemization of the compound, and the second one being the weakness of the carbon-Se (C—Se) bond towards harsh conditions. Taking those into consideration, and in accordance with an aspect of the present disclosure, the synthesis was started using the commercially available L-histidine methyl-ester 1 (Scheme 2).

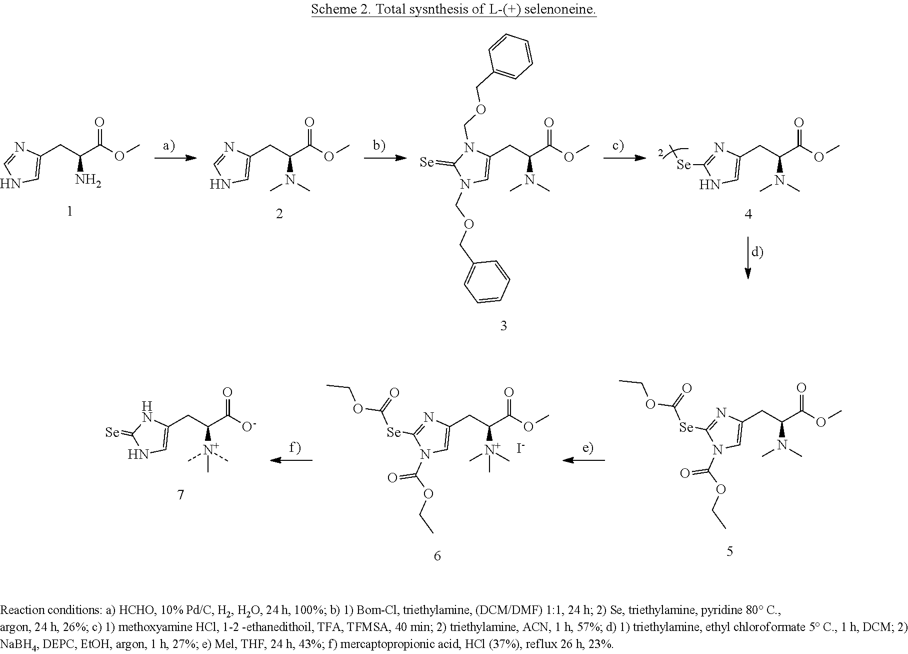

The α-amino group of the L-histidine methyl-ester 1 starting material was dimethylated with formaldehyde over a palladium-on-charcoal (Pd/C) catalyst under hydrogenolytic conditions to give the N,N-dimethyl-L-histidine methyl ester 2. This manner of introducing the methyl groups has the advantage of giving a quantitative yield relative to other reductive amination procedures using NaCNBH₃ (Lim et al., 2019).^{[14]} It is advantageously performed prior to the introduction of Se to avoid poisoning the catalyst and to avoid the loss of Se.^{[15]} The next step was the introduction of Se at C-2 of the imidazole moiety. To achieve this, the alkylation of both nitrogen atoms of the imidazole moiety, as exemplified by the use of an amino protecting group, opens the route for a selective deprotonation at C-2 under basic conditions, advantageously providing for the selenation reaction to take place. In order to avoid racemization due to the acidity of the α-carbon, a stable alkylating agent that can be deprotected under acidic conditions while also being compatible with the selenone group was used. To that effect, benzyl chloromethyl ether (Bom-Cl) was advantageously used. The resulting N,N′-bis-alkylated imidazolium derivative was then reacted with triethylamine and elemental selenium in pyridine, to give the selone intermediate 3 (26%). It should be noted that ring opening using the Bamberger reaction, followed by cyclization using potassium selenocyanate (KSeCN), failed to afford the desired selone intermediate. However, the analogous thione intermediate could be formed using KSCN, as illustrated in the synthesis of ergothioneine.^{[16]} A synthetic approach comprising the direct selenation via a radical pathway using diaryl diselenide^{[17]} was also attempted but failed to provide the desired selone intermediate 3 (the reaction failing at the Se-deprotection step).

Selone intermediate 3 was subsequently deprotected in trifluoroacetic acid (TFA) using an excess of trifluoromethanesulfonic acid (TFMSA) in the presence of a scavenger. Surprisingly, the selenone functionality was found to be resistant to the harsh acidic conditions. Precipitation, followed by desalting, afforded the oxidized (dimer) form of N,N-dimethyl-seleno-histidine methyl ester 4 (57%). The imidazole N—H of intermediate 4 was subsequently protected using ethyl chloroformate under basic conditions, followed by treatment with sodium borohydride (NaBH₄) and diethyl pyrocarbonate (DEPC) to provide intermediate 5 (27%). The latter transformation was advantageously performed in two separate steps, instead of a “one-pot” reaction using ethyl chloroformate, to avoid regenerating the oxidized (dimer) form following the reduction and thus compromising the Se protection. Quaternization of the tertiary amine of intermediate 5 using iodomethane afforded intermediate 6 (43%). Finally, global deprotection while avoiding racemization, by acidic hydrolysis using concentrated HCl under reflux, afforded the desired L-(+)-selenoneine (7). Circular dichroism confirmed the positive optically active nature of the compound 7 (FIG. 2), and the spectroscopic data matched that of the natural isolated product.^{[18]}

### Example 3—General Synthetic Approach to Selenoneine Synthesis

In order to improve on the overall yield of the selenoneine product and advantageously provide for “scale-up” conditions, a further general synthetic approach is illustrated starting from both racemic (Scheme 3) and enantiomeric (Scheme 4) histidine alkyl ester.

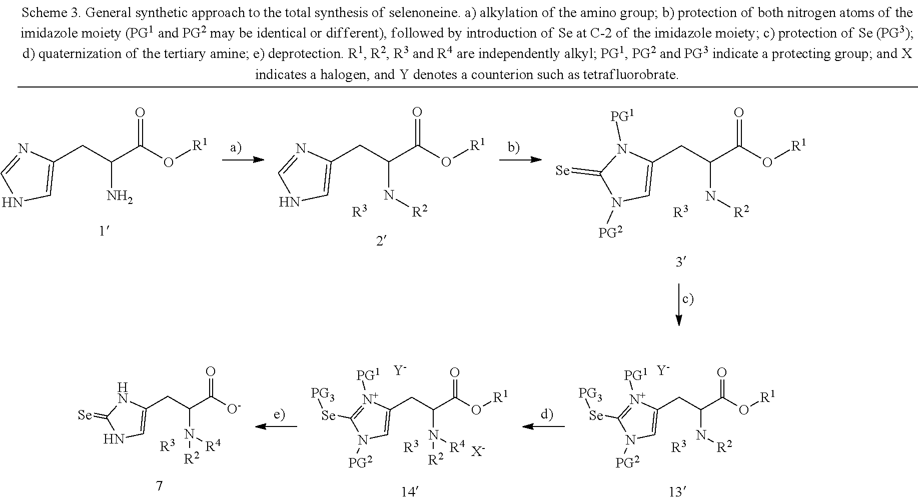

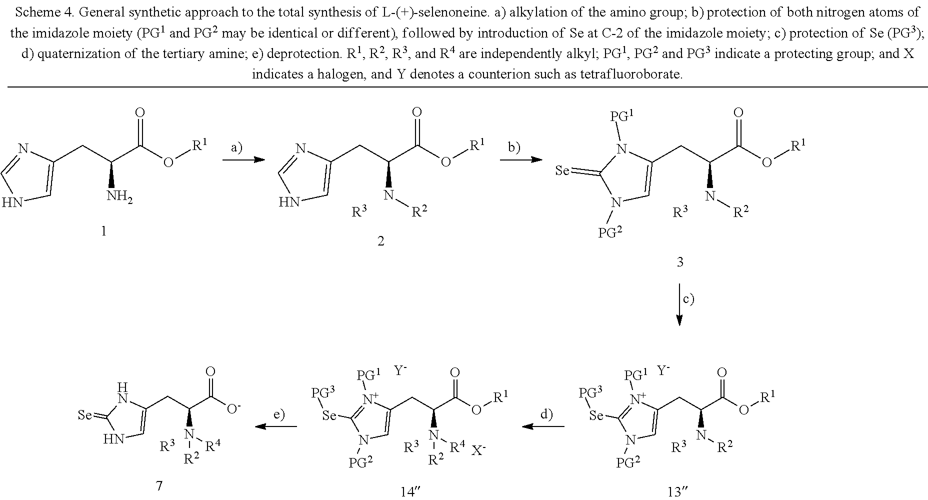

### Example 4—L-(+)-Selenoneine Synthesis

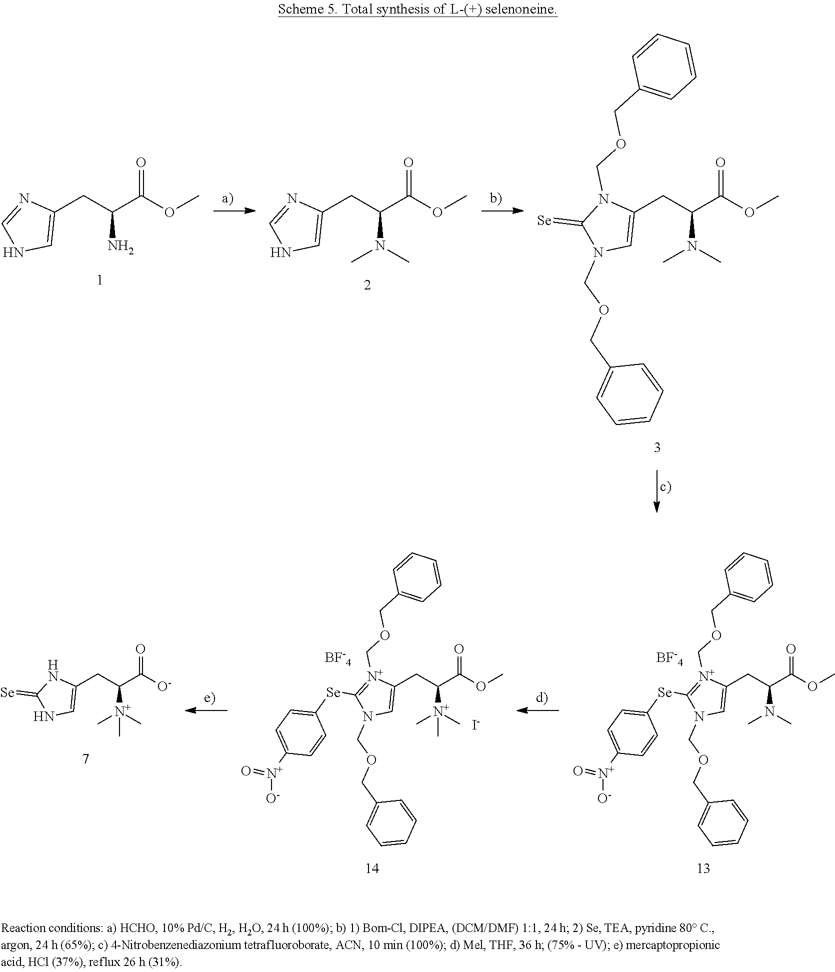

For the “scale-up” conditions, three major aspects were taken into account, namely: the use of a super acid (trifluoromethanesulfonic acid; TFSMA); the number of steps; and the overall yield. Accordingly, steps (b), (c) and (d) of Scheme 2 were targeted. The aim was to improve the yield of step (b) and then try to protect the Se of compound 3 without having to remove the protecting groups already in place on both amines of the imidazole moiety. This advantageously provides for the elimination of the deprotection step using TFSMA, while providing for the direct alkylation of the tertiary amine with the Se and imidazole amines remaining protected.

As illustrated in Scheme 5, the yield of step (b) was improved from 26% to 65%. This improvement was attributed to two modifications made relative to Scheme 2. The first modification being the replacement of triethylamine (TEA) with diisopropylethylamine (DIPEA) which is sterically more hindered for the alkylation of the imidazole amines using Bom-Cl prior to the selenation reaction, and the second modification being the increase (e.g., doubling) of the amount of selenium used in the selenation reaction. TEA was observed scavenging the alkylating agent in view of its alkylation by Bom-Cl. The selone intermediate 3 was then reacted with 4-nitrobenzenediazonium tetrafluoroborate in acetonitrile (ACN) to afford intermediate 13 in quantitative yield without the need for further purification. It should be noted that diazonium salts (e.g., 4-nitrobenzenediazonium tetrafluoroborate) were advantageously used to provide intermediate 13. Quaternization of the tertiary amine of intermediate 13 using iodomethane afforded intermediate 14 (75%). It should be noted that intermediate 14 is somewhat unstable and has the propensity of losing a Bom protecting group (i.e., PG¹ as illustrated in Schemes 3 and 4). Both intermediate 14, and intermediate 14A having lost the PG¹ protecting group, can be readily converted into the desired selenoneine product. Finally, global deprotection while avoiding racemization, by acidic hydrolysis using concentrated HCl under reflux, afforded the desired L-(+)-selenoneine (7). The overall yield of compound 7 was 15% relative to 2% following the synthetic route depicted in Scheme 2.

### Example 5—General Synthetic Approach to Iso-Selenoneine and Poly-Seleno-Hercynine Synthesis

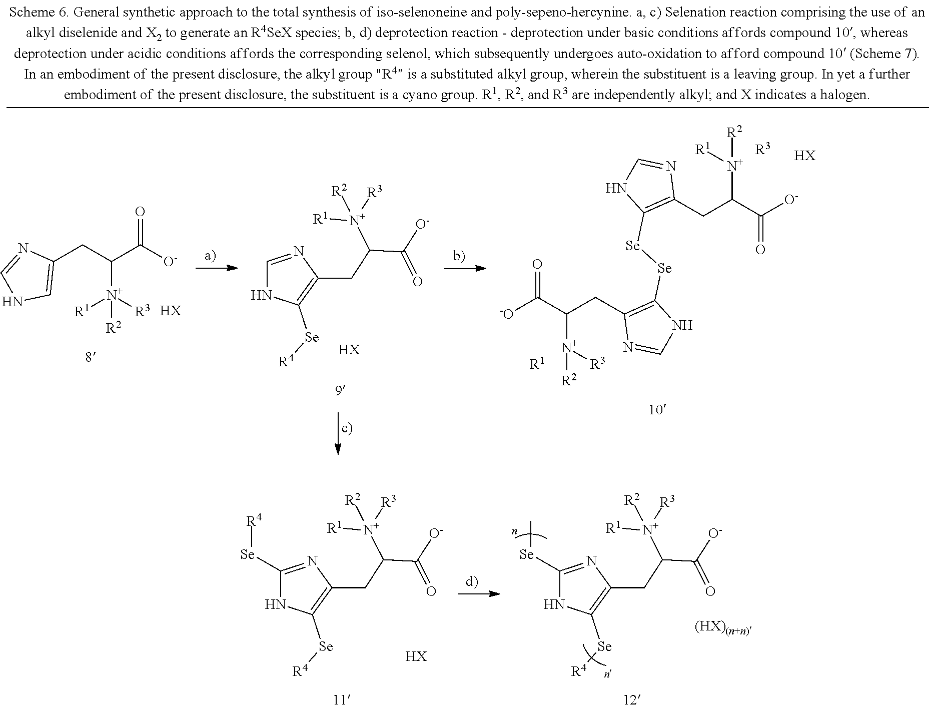

### Example 6—Deprotection Under Basic and Acidic Conditions

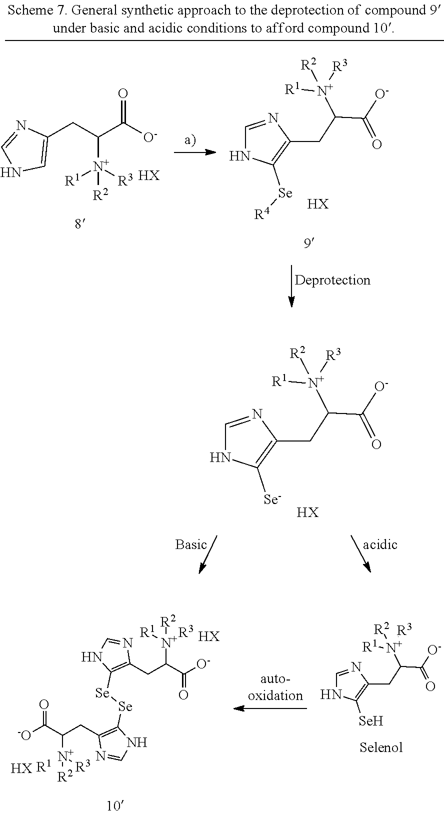

### Example 7—Iso-Selenoneine and Poly-Seleno-Hercynine Synthesis

A rapid and efficient synthetic pathway for introducing Se at C-5, and subsequently at C-2 of the imidazole moiety of hercynine was serendipitously discovered. The synthetic pathway relies on the direct selenation of commercially available hercynine by initially generating an electrophilic R—Se—I species from the reaction between a diselenide and iodine (I₂). The R—Se—I species subsequently undergoes reaction with the imidazole moiety of hercynine to afford the corresponding C-5 seleno-substituted derivative. The latter may subsequently undergo a second substitution at C-2 to yield the C5-C2 di-seleno-substituted derivative (Scheme 8). In an embodiment of the present disclosure, the corresponding C-5 seleno-substituted derivative undergoes dimerization through a subsequent β-elimination reaction.

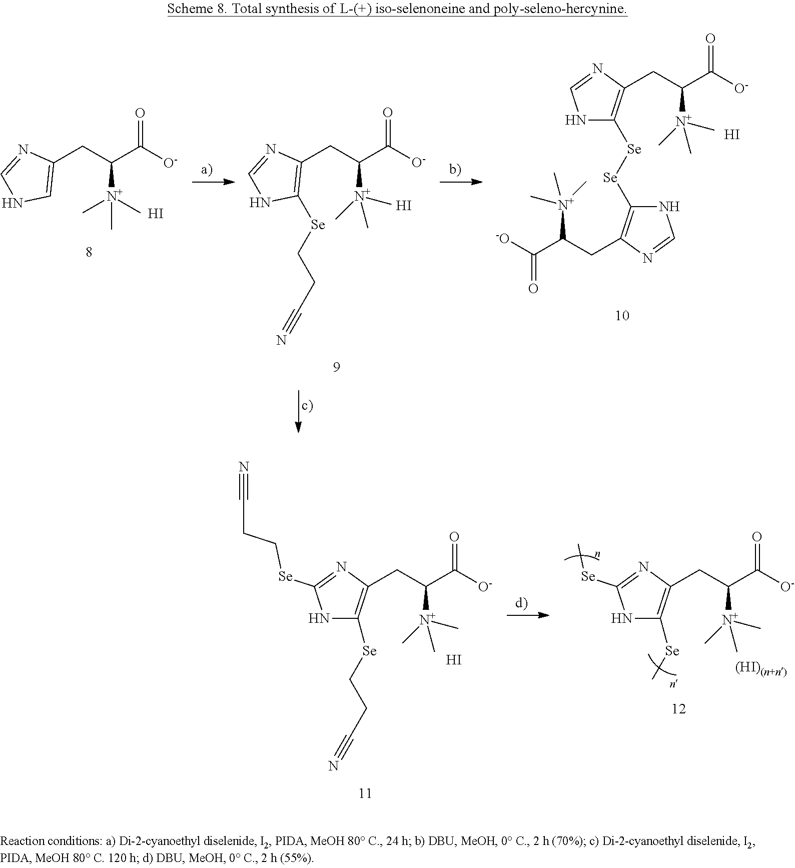

As illustrated in Scheme 8, commercially available hercynine was reacted with di-2-cyanoethyl diselenide and a catalytic amount of I₂, as well as (diacetoxyiodo)benzene, which serves as an oxidant to regenerate I₂ from the liberated hydrogen iodide (HI). The reaction was monitored by HPLC-MS-PDA. During the first 24 h, intermediate 9 was the major compound, after which a small amount of intermediate 11 could be observed. The mixture was allowed to react for 120 h affording intermediates 9 (10%) and 11 (2.5%) respectively, which could be separated by preparative HILIC-HPLC due to their respective high polarities. Intermediate 9 was subsequently treated with DBU to remove the cyanoethyl protecting group, affording iso-selenoneine 10 (70%) following a subsequent β-elimination reaction. It is surmised that in view of the Se being attached at C-5 instead of C-2, iso-selenoneine 10 may possess different attributes in terms of antioxidant activity and methylmercury detoxification relative to L-(+)-selenoneine 7. Indeed, a comparison of ergothioneine with the ovothiols is indicative of a greater antioxidant activity for the ovothiols due to a lower pKa value of 4.7 for S at C-5 versus 8.7 for S at C-2.^{[19]} Finally, removal of the cyanoethyl protecting groups from intermediate 11 by treatment with DBU afforded poly-seleno-hercynine 12 (55%). Attempts to fully reduce the polymer to its monomeric form have as yet been unsuccessful, due to the propensity of the resulting selenols to rapid oxidation.

### In-Vitro Activity Assessment

Since selenoneine is the analog of the well-known natural antioxidant and cytoprotectant ergothioneine, its capacity as an antioxidant and cytoprotectant was assessed. To that effect, the capacity of selenoneine to protect cells against the cytotoxic effects induced by t-butyl hydroperoxide (t-BuOOH) and methylmercury chloride (MeHgCl) was assessed. Human erythroid K562 cells expressing the ergothioneine transporter (OCTN1) were exposed to the IC₅₀ values of t-BuOOH and MeHgCl respectively, in the presence of increasing concentrations of either selenoneine or ergothioneine. The cell viability was subsequently assessed using the trypan blue viability exclusion test.^{[20]}

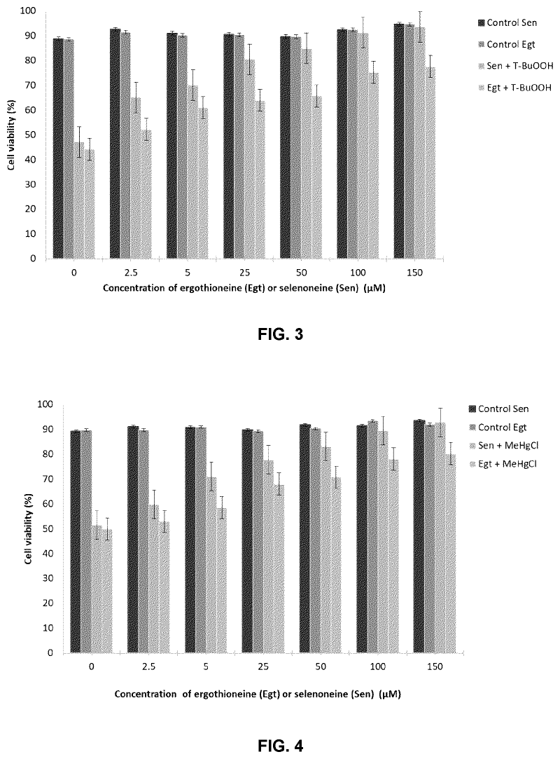

The viability of cells exposed to t-BuOOH increased with increasing concentrations of either selenoneine or ergothioneine (FIG. 3). Importantly, selenoneine exhibited a greater protective effect relative to ergothioneine against t-BuOOH induced cytotoxicity. Treatment with selenoneine, at concentrations of 100 μM or higher, resulted in cell viabilities comparable to those of the control groups. These results illustrate the superior protective effect of selenoneine against t-BuOOH induced oxidative stress relative to ergothioneine.

The protective effect of selenoneine and ergothioneine against the cytotoxic effects induced by methylmercury chloride (MeHgCl) in human erythroid K562 cells is illustrated in FIG. 4. The viability of cells exposed to MeHgCl increased with increasing concentrations of either selenoneine or ergothioneine. Importantly, selenoneine exhibited a greater protective effect relative to ergothioneine against MeHgCl induced cytotoxicity. Treatment with selenoneine, at concentrations of 100 μM or higher, resulted in cell viabilities comparable to those of the control groups (not treated with MeHgCl; pretreatment with the protective agents only). Treatment with ergothioneine, at concentrations of 150 μM or higher, did not result in cell viabilities comparable to those of the control groups. These results illustrate the superior protective effect of selenoneine against MeHgCl induced oxidative stress relative to ergothioneine. It is believed that the MeHgCl toxicity, even though not completely understood, is partly due to a redox imbalance resulting from interactions between selenol and thiol groups in small molecules and enzymes.^{[21]}

### General Methods and Materials

Reagents and solvents were obtained from commercial suppliers (Sigma-Aldrich, TCl Chemicals, Alfa Aesar) and used without further purification, unless otherwise noted. Di-2-cyanoethyl diselenide and hercynine HI were provided by the Organic Synthesis Service, Medicinal Chemistry Platform, Centre de Recherche du Centre Hospitalier Universitaire (CHU) de Québec-Université Laval. Reaction progress was monitored by thin layer chromatography (TLC), using EMD silica gel 60 F254 aluminum plates (E. Merck; Darmstadt, Germany). Spots could be visualized with UV light (254 nm), and/or followed by staining using a potassium bismuth iodide solution (Dragendorff's reagent), or a cerium ammonium molybdate (CAM) solution or a potassium-permanganate solution, followed by heating on a hot plate. SiliCycle® R10030B 230-400 mesh silica gel (SiliCycle Inc., Québec, QC, Canada) was used for flash chromatography. Preparative high-performance liquid chromatography (H PLC) analyses were performed on a Shimadzu Prominence instrument (Shimadzu Corporation, Kyoto, Japan) equipped with a HILIC preparative column (Waters, Milford, MA). Nuclear magnetic resonance (NMR) spectra were recorded on Bruker Avance 400 and Ascend 300 digital spectrometers (Billerica, MA, USA). High-resolution mass spectra (HRMS) were recorded on an Acquity UPLC coupled to a Xevo G2-XS ESI-QTof MS (Waters, Milford, MA). Finally, circular dichroism and optical rotation were recorded on a JASCO J-815 and a DIP-370 instrument respectively.

In vitro protection assays against either methylmercury chloride (MeHgCl) or tert-butyl hydroperoxide (t-BuOOH) were conducted with the K562-S human leukemia cell line (REF #CRL-3343™) obtained from the American Type Culture Collection (ATCC). Cells were cultured in Gibco RPMI 1640 complete growth medium with L-Glutamine (REF #31800-105), NaHCO₃ (2 g/L), 10% FBS (REF #080-750), 2% antibiotic-antimycotic from Gibco (REF #15240-062), pH 7.4, and maintained at 37° C. in a 5% CO₂ incubator and protected from light.

An uptake induction step, in a saline buffer containing 125 mM of NaCl; 25 mM of HEPES-NaOH (pH 7.4); 5.6 mM of D(+)-glucose; 4.8 mM KCl; 1.2 mM of KH₂PO₄; 1.2 mM of CaCl₂; and 1.2 mM of MgSO₄, was performed prior to adding selenoneine or ergothioneine to the cell medium.^{[22,23]} Cells were resuspended in triplicate and pre-incubated in the saline buffer at a density of 2×10⁶ cells/mL for 20 minutes at 37° C. under slight agitation. Cells were then incubated during 4 hours in the same buffer and conditions but supplemented with the protective agent of interest—either ergothioneine or selenoneine—in the presence of 0.15 mM of reduced glutathione (GSH). Concentrations of protective agents, either ergothioneine or selenoneine, used for these experiments were 2.5, 5, 25, 50, 100, and 150 μM (FIGS. 3 and 4). Cells were then washed twice in cold phosphate-buffered saline (PBS) and resuspended in complete RPMI growth medium and divided into two groups at a density of 1×10⁶ cells/mL. One group of cells remained untreated (control group) while the other group was treated with either 830 μM of t-BuOOH, or 5 μM of MeHgCl. These concentrations were determined to decrease cell survival by 50% in preliminary experiments (IC₅₀ values). Cells were then incubated at 37° C. under slight agitation over a period of one (1) hour in the case of t-BuOOH, or overnight (18h) for MeHgCl. The cells were then washed twice in cold PBS before being resuspended in complete RPMI growth medium at a density of 5×10⁵ cells/mL. The trypan blue viability exclusion test was then conducted using a Hausser Scientific™ Bright-Line™ Phase Hemacytometer. Viable cells (%)=[total number of viable cells per mL of aliquot/total number of cells per mL of aliquot]×100.^{[20]} Results are expressed as the mean±standard error of the mean of triplicates. These experiments were repeated twice, and similar results were obtained.

### Compound Characterization

### Methyl (2S)-2-(dimethylamino)-3-(1H-imidazol-5-yl)propanoate dihydrochloride (2)

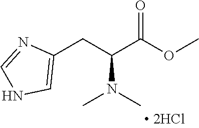

To a solution of L-histidine methyl ester dihydrochloride (1) (5.0 g, 20.7 mmol) in 60 mL of deionized water was added formaldehyde (4.0 mL of a 37% w/v aq. solution, 49.7 mmol). The mixture was then hydrogenated under hydrogen pressure in the presence of 10% Pd/C (1 g) for 24 h. Upon reaction completion, monitored by ¹H-NMR, the mixture was filtered through a pad of Celite, washed with water and concentrated under vacuum to yield the title compound as a yellowish-white solid (5.6 g, quant), which was used in the next step without further purification. ¹H NMR (400 MHz, D₂O) δ8.70 (s, 1H), 7.45 (s, 1H), 4.55 (dd, J=9.6, 5.0 Hz, 1H), 3.80 (s, 3H), 3.56 (dd, 2H, J=14.5, 7.9 Hz), 3.04 (s, 6H) ppm; HRMS (ESI-QTof) calcd. for C₉H₁₅N₃O₂ (M+H⁺) m/z=198.1243, found 198.1248.

### Methyl (S)-3-(1,3-bis((benzyloxy)methyl)-2-selenoxo-2,3-dihydro-1H-imidazol-4-yl)-2-(dimethylamino)propanoate (3) (as per Scheme 2)

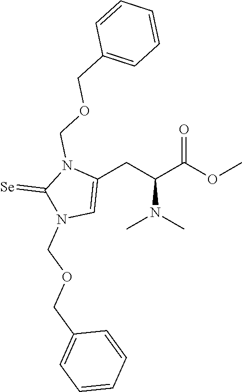 [SMILES: COC(=O)[C@H](Cc1cn(COCc2ccccc2)c(=[Se])n1COCc1ccccc1)N(C)C]

To an ice-cooled solution of methyl (2S)-2-(dimethylamino)-3-(1H-imidazol-5-yl)propanoate dihydrochloride (2) (5.0 g, 18.5 mmol) in 60 mL DCM/DMF (1:1) was added dropwise triethylamine (12.8 mL, 92.5 mmol) and benzyl chloromethyl ether (6.05 mL, 44.4 mmol). The reaction mixture was allowed to stir at room temperature (i.e., 20-22° C.) for 24 h, at which time, the mixture was filtered, washed with brine, dried over MgSO₄, and concentrated under vacuum. The residue was further dissolved in 60 mL of anhydrous pyridine, cooled to 0° C., and triethylamine (8.3 mL, 60 mmol) and elemental Se (1.7 g, 18.5 mmol) were then added to the reaction mixture, which was subsequently stirred at 80° C. under argon for 24 h. After this period, the reaction mixture was filtered, washed with brine, and concentrated under vacuum. Purification of the residue by flash chromatography on silica gel (DCM/EtOAc, 10:1) gave the title compound 3 (2.55 g, 26% over two steps) as a yellow oil. ¹H NMR (400 MHz, CDCl3) δ7.33 (p, J=8.5 Hz, 10H), 6.87 (s, 1H), 5.89 (d, J=11.0 Hz, 1H), 5.78 (d, J=10.8 Hz, 1H), 5.67 (s, 2H), 4.73 (s, 2H), 4.64 (s, 2H), 3.67 (s, 3H), 3.47 (t, J=7.4 Hz, 1H), 3.06 (dd, J=15.8, 7.9 Hz, 1H), 2.91 (dd, J=15.8, 7.0 Hz, 1H), 2.29 (s, 6H) ppm; HRMS (ESI-QTof) calcd. for C₂₅H₃₁N₃O₄Se (M+H⁺) m/z=518.1559, found 518.1555.

### Dimethyl 3,3′-(diselanediylbis(1H-imidazole-2,4-diyl))(2S,2′S)-bis(2-(dimethylamino)propanoate) (4)

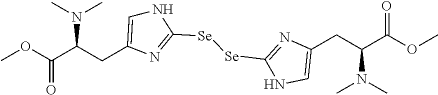

To an ice-cooled flask containing methyl (S)-3-(1,3-bis((benzyloxy)methyl)-2-selenoxo-2,3-dihydro-1H-imidazol-4-yl)-2-(dimethylamino)propanoate (3) (2.45 g, 4.74 mmol) and methoxyamine hydrochloride (3.96 g, 47.4 mmol) was slowly added 25 mL of TFA followed by the addition of 1,2-ethanedithiol (7.76 mL, 94.8 mmol). The reaction mixture was allowed to stir for 15 min at 0° C. at which time TFMSA (6 mL) was added dropwise over a 15-min period. The reaction mixture was then stirred at rt for 40 min, and cold diethyl ether was then added to precipitate the crude product. The precipitate was washed with cold diethyl ether and DCM, dissolved in a minimum volume of MeOH, and cold diethyl ether was added again. The final precipitate was washed with diethyl ether and dried under vacuum to give the title product in the form of a 4×TFMSA salt (1.81 g, 66%). The product was desalted to give the free form as follows: the salt (1.81 g, 1.57 mmol) was dissolved in 50 mL ACN, then cooled to 0° C., and triethylamine (2.32 mL, 16.92 mmol) was added dropwise. The reaction mixture was stirred for 1 h at rt and then concentrated under vacuum. The residue was dissolved in 50 mL of DCM, washed with brine, dried over MgSO₄, and concentrated under vacuum to yield to title compound (750 mg, 57%) as a yellow solid. ¹H NMR (400 MHz, CD₃CN) δ 6.97 (s, 2H), 3.63 (s, 6H), 3.56 (t, J=7.6 Hz, 2H), 3.05 (dd, J=14.9, 8.1 Hz, 2H), 2.93 (dd, J=14.8, 7.3 Hz, 2H), 2.34 (s, 12H) ppm; HRMS (ESI-QTof) calcd. for C₁₈H₂₈N₆O₄Se₂ (M+H⁺) m/z=553.0585, found 553.0587.

### Ethyl (S)-4-(2-(dimethylamino)-3-methoxy-3-oxopropyl)-2-((ethoxycarbonyl)selanyl)-1H-imidazole-1-carboxylate (5)

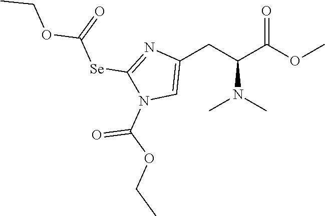 [SMILES: CCOC(=O)[Se]c1nc(C[C@@H](C(=O)OC)N(C)C)cn1C(=O)OCC]

To an ice-cooled solution of dimethyl 3,3′-(diselanediylbis(1H-imidazole-2,4-diyl))(2S,2′S)-bis(2-(dimethylamino)propanoate) (4) (600 mg, 1.1 mmol) in DCM (15 mL) was added triethylamine (1.5 mL, 11 mmol) and ethyl chloroformate (0.45 mL, 4.77 mmol). The reaction mixture was stirred for 1 h at 5° C. and then concentrated under vacuum. The residue was subsequently dissolved in 50 mL of DCM, washed with brine, dried over MgSO₄, and concentrated under vacuum. The residue was subsequently dissolved in anhydrous EtOH (10 mL) under argon followed by the addition of NaBH₄ (83.2 mg, 2.2 mmol), and the reaction mixture was stirred for 15 min, at which point, diethylpyrocarbonate (DEPC) (0.32 mL, 2.2 mmol) was added. After 1 h, water (150 mL) was added, and the reaction mixture was extracted 3 times with EtOAc; the combined organic extracts were subsequently concentrated under vacuum. Purification of the residue by flash chromatography on silica gel (cyclohexane/EtOAc, 1:1) gave the title compound (250 mg, 27% over two steps) as a yellow oil. ¹H NMR (400 MHz, CDCl₃) δ 7.38 (s, 1H), 4.42 (q, J=7.4 Hz, 2H), 4.32 (q, J=7.0 Hz, 2H), 3.67 (s, 3H), 3.02 (dd, J=14.6, 8.6 Hz, 1H), 2.90 (dd, J=14.6, 6.5 Hz, 1H), 2.36 (s, 6H), 1.41 (t, J=7.1 Hz, 3H), 1.31 (t, J=7.2 Hz, 3H) ppm; HRMS (ESI-QTof) calcd. for C₁₅H₂₃N₃O₆Se (M+H⁺) m/z=422.0831, found 422.0826.

### (S)-3-(1-(ethoxycarbonyl)-2-((ethoxycarbonyl)selanyl)-1H-imidazol-4-yl)-1-methoxy-N,N,N-trimethyl-1-oxopropan-2-aminium iodide (6)

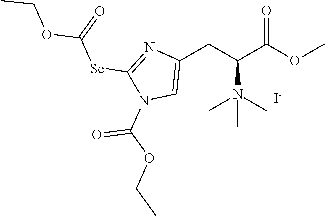 [SMILES: CCOC(=O)[Se]c1nc(C[C@@H](C(=O)OC)[N+](C)(C)C)cn1C(=O)OCC ; [I-]]

To a solution containing ethyl (S)-4-(2-(dimethylamino)-3-methoxy-3-oxopropyl)-2-((ethoxycarbonyl)selanyl)-1H-imidazole-1-carboxylate (5) (226.5 mg, 0.54 mmol) in 5 mL of dry THF was added iodomethane (65 μL, 1.05 mmol), and the reaction mixture was stirred at rt for 24 h. After that time, the precipitate was filtered, rinsed with THF, and then dried under vacuum to yield the title compound (127 mg, 43%) as a white solid. ¹H NMR (400 MHz, CDCl3) δ 7.75 (s, 1H), 5.03 (dd, J=8.4, 4.9 Hz, 1H), 4.45 (q, J=7.2 Hz, 2H), 4.33 (q, J=7.0 Hz, 2H), 3.81 (s, 3H), 3.69 (d, J=4.9 Hz, 1H), 3.64 (s, 9H), 3.38 (dd, J=15.0, 8.5 Hz, 1H), 1.44 (t, J=7.1 Hz, 3H), 1.33 (t, J=7.2 Hz, 3H) ppm; HRMS (ESI-QTof) calcd. for C₁₅H₂₅N₃O₆Se (M+) m/z=436.0988, found 436.0991.

### (S)-3-(2-selenoxo-2,3-dihydro-1H-imidazol-4-yl)-2-(trimethylammonio) propanoate (7)

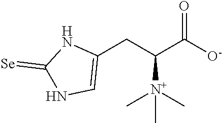

To a flask containing (S)-3-(1-(ethoxycarbonyl)-2-((ethoxycarbonyl)selanyl)-1H-imidazol-4-yl)-1-methoxy-N,N,N-trimethyl-1-oxopropan-2-aminium iodide (6) (100 mg, 0.182 mmol) and 3-mercaptopropionic acid (1.25 mL, 1.5 mmol) was added 5 mL of concentrated HCl (36%) and the reaction mixture was allowed to stir under reflux for 26 h. Following solvent removal under vacuum, 20 mL of water were added, and the resulting mixture extracted with 3× (50 mL) EtOAc. The pH of the aqueous solution was adjusted to 6-7 with an aqueous solution of NH₄OH (7%) and then evaporated to dryness. The purification of the residue by a semi-preparative HPLC gave the title compound (12 mg, 23%) as yellow solid. ¹H NMR (500 MHz, D₂O) δ6.85 (s, 1H), 3.75 (dd, J=11.8, 3.8 Hz, 1H_{α}), 3.16 (m, 1H_{β}), 3.13 (s, 9H), 3.07 (m, 1H_{β′}) ppm; ¹³C NMR (126 MHz, D₂O) δ170.25, 138.28, 129.87, 118.68, 77.53, 52.05, 23.70 ppm; HRMS (ESI-QTof) calcd. for C₉H₁₅N₃O₂Se (M+H⁺) m/z=278.0408, found 278.0410.

### (S)-3-(5-((2-cyanoethyl)selanyl)-1H-imidazol-4-yl)-2-(trimethylammonio) propanoate hydroiodide (9)

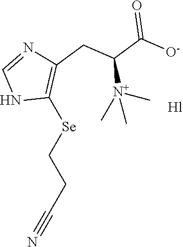 [SMILES: C[N+](C)(C)[C@@H](Cc1nc[nH]c1[Se]CCC#N)C(=O)[O-]]

To a solution containing di-2-cyanoethyl diselenide (804 mg, 3 mmol) and I₂ (85 mg, 0.75 mmol) in 20 ml of MeOH, was added hercynine HI (487.5 mg, 1.5 mmol) and PIDA (145 g, 4.5 mmol). The reaction mixture was refluxed at 80° C. for 120 h at which time the solution was cooled down to rt, filtered, and then the solvent evaporated under vacuum. The residue was subsequently dissolved in 50 mL of water and extracted 3 times with EtOAc. The aqueous fraction was evaporated to dryness, the residue dissolved in a minimum volume of MeOH, and the resulting solution processed through preparative-HPLC to yield the title compound 9 (68 mg, 10%) as a yellow oil. ¹H NMR (400 MHz, D₂O) δ7.92 (s, 1H), 3.90 (dd, J=10.1, 5.1 Hz, 1H), 3.41-3.34 (m, 2H), 3.29 (s, 9H), 2.93 (ddq, J=32.5, 12.3, 6.5 Hz, 2H), 2.78 (dq, J=17.4, 8.1 Hz, 2H) ppm; ¹³C NMR (101 MHz, MeOD) δ 167.62, 136.09, 134.23, 117.61, 113.19, 76.27, 49.59, 22.98, 20.96, 16.44 ppm; HRMS (ESI-QTof) calcd. for C₁₂H₁₈N₄O₂Se (M+H⁺) m/z=331.0674, found 331.0675.

### (2S,2′S)-3,3′-(diselanediylbis(1H-imidazole-5,4-diyl))bis(2-(trimethylammonio) propanoate) hydroiodide (10)

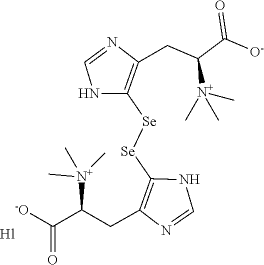

To an ice-cooled solution of (S)-3-(5-((2-cyanoethyl)selanyl)-1H-imidazol-4-yl)-2-(trimethylammonio)propanoate hydroiodide (9) (68 mg, 0.148 mmol) in 5 mL MeOH was added an excess of DBU (2 mL, 13.36 mmol) and the reaction was allowed to stir at 0° C. for 2 h. ACN was subsequently added to the mixture to initiate precipitation. The precipitate was filtered out, washed with ACN, and allowed to dry under vacuum. The compound was then dissolved in a minimum volume of ACN and water. Preparative HPLC followed by lyophilisation yielded the title compound as a yellow solid (35.8 mg, 60%). ¹H NMR (400 MHz, D₂O) δ8.07 (s, 2H), 3.71 (d, J=11.8 Hz, 2H), 3.18 (s, 19H), 2.92-2.61 (m, 4H) ppm; ¹³C NMR (101 MHz, D₂O) δ172.08, 140.75, 138.91, 118.22, 79.56, 54.07, 25.82 ppm; HRMS (ESI-QTof) calcd. for C₁₈H₂₈N₆O₄Se₂ (M+2H⁺) m/z=277.0331, found 277.0336.

### (S)-3-(2,5-bis((2-cyanoethyl)selanyl)-1H-imidazol-4-yl)-2-(trimethylammonio) propanoate hydroiodide (11)

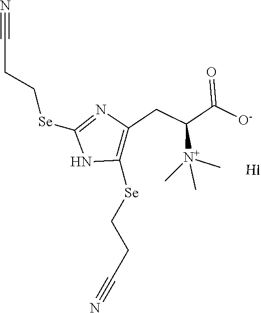 [SMILES: C[N+](C)(C)[C@@H](Cc1nc([Se]CCC#N)[nH]c1[Se]CCC#N)C(=O)[O-]]

Compound 11 was obtained in accordance with the procedure described herein for the preparation of compound 9 in 2.5% yield. ¹H NMR (400 MHz, D₂O) δ3.89 (dd, J=10.4, 4.9 Hz, 1H), 3.44-3.32 (m, 3H), 3.29 (s, 9H), 3.16 (t, J=6.7 Hz, 2H), 3.05-2.93 (m, 1H), 2.93-2.75 (m, 5H) ppm; ¹³C NMR (101 MHz, D₂O) δ170.20, 139.15, 133.36, 120.58, 120.11, 117.08, 77.85, 52.08, 24.61, 22.78, 22.68, 19.44, 18.82 ppm; HRMS (ESI-QTof) calcd. for C₁₅H₂₁N₅O₂Se₂ (M+) m/z=464.0111, found 464.0107.

### Methyl (S)-3-(1,3-bis((benzyloxy)methyl)-2-selenoxo-2,3-dihydro-1H-imidazol-4-yl)-2-(dimethylamino)propanoate (3) (as per Scheme 5)

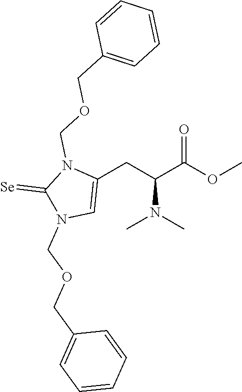 [SMILES: COC(=O)[C@H](Cc1cn(COCc2ccccc2)c(=[Se])n1COCc1ccccc1)N(C)C]

To an ice-cooled solution of methyl (2S)-2-(dimethylamino)-3-(1H-imidazol-5-yl)propanoate dihydrochloride (2) (8.0 g, 29.6 mmol) in 100 mL (DCM/DMF, 1:1) was added dropwise diisopropylamine (25.3 mL, 148.0 mmol) and benzyl chloromethyl ether (10.0 mL, 71.0 mmol). The reaction mixture was allowed to stir at room temperature (i.e., 20-22° C.) for 24 h, at which time, the mixture was filtered, washed with brine, dried over MgSO₄, and concentrated under vacuum. The residue was further dissolved in 60 mL of anhydrous pyridine, cooled to 0° C., and triethylamine (13.3 mL, 96.1 mmol) and elemental Se (4.7 g, 59.2 mmol) were then added to the reaction mixture, which was subsequently stirred at 80° C. under argon for 24 h. After this period, the reaction mixture was filtered, washed with brine, and concentrated under vacuum. Purification of the residue by flash chromatography on silica gel (DCM/EtOAc, 10:1) gave the title compound 3 (9.90 g, 65% over two steps) as a yellow oil. ¹H NMR (400 MHz, CDCl3) δ7.33 (p, J=8.5 Hz, 10H), 6.87 (s, 1H), 5.89 (d, J=11.0 Hz, 1H), 5.78 (d, J=10.8 Hz, 1H), 5.67 (s, 2H), 4.73 (s, 2H), 4.64 (s, 2H), 3.67 (s, 3H), 3.47 (t, J=7.4 Hz, 1H), 3.06 (dd, J=15.8, 7.9 Hz, 1H), 2.91 (dd, J=15.8, 7.0 Hz, 1H), 2.29 (s, 6H) ppm; HRMS (ESI-QTof) calcd. for C₂₅H₃₁N₃O₄Se (M+H⁺) m/z=518.1559, found 518.1556.

### (S)-1,3-bis((benzyloxy)methyl)-4-(2-(dimethylamino)-3-methoxy-3-oxopropyl)-2-((4-nitrophenyl)selanyl)-1H-imidazol-3-ium tetrafluoroborate (13)

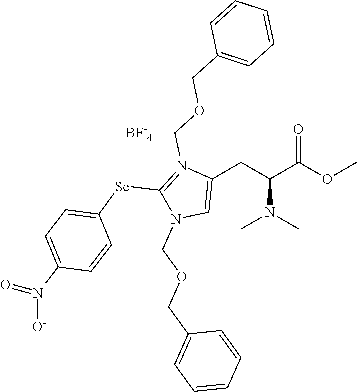 [SMILES: COC(=O)[C@H](Cc1cn(COCc2ccccc2)c([Se]c2ccc([N+](=O)[O-])cc2)[n+]1COCc1ccccc1)N(C)C ; "BF-4"]

To a solution containing methyl (S)-3-(1,3-bis((benzyloxy)methyl)-2-selenoxo-2,3-dihydro-1H-imidazol-4-yl)-2-(dimethylamino)propanoate (3) (50 mg, 0.1 mmol) in 2.5 ml of ACN, was added dropwise a solution containing 4-nitrobenzenediazonium tetrafluoroborate (26.1 mg, 0.11 mmol) in 5 mL of ACN. The reaction mixture was allowed to stir for 30 min and then the solvent was removed under vacuum to afford the title compound quantitatively as a yellow brownish solid (72.5 mg, quant). ¹H NMR (300 MHz, CD₃CN) δ 8.01 (m, 2H), 7.85 (s, 1H), 7.54 (m, 2H), 7.29 (m, 10H), 5.77 (d, J=17.3 Hz, 4H), 4.59 (d, J=11.2 Hz, 4H), 3.90 (t, J=7.4 Hz, 1H), 3.76 (s, 3H), 3.30 (m, 2H), 2.53 (s, 6H); HRMS (ESI-QTof) calcd. for C₃₁H₃₅N₄O₆Se (M⁺) m/z=639.1721, found 639.1724.

Large scale: To a solution containing methyl (S)-3-(1,3-bis((benzyloxy)methyl)-2-selenoxo-2,3-dihydro-1H-imidazol-4-yl)-2-(dimethylamino)propanoate (3) (6.2 g, 12 mmol) in 30 ml of ACN, was added dropwise a solution containing 4-nitrobenzenediazonium tetrafluoroborate (3.12 g, 13.2 mmol) in 60 mL of ACN. The reaction mixture was allowed to stir for 30 min and then the solvent was removed under vacuum to yield the title compound quantitatively as a yellow brownish solid (8.7 g, quant).

### (S)-1,3-bis((benzyloxy)methyl)-4-(3-methoxy-3-oxo-2-(trimethylammonio) propyl)-2-((4-nitrophenyl)selanyl)-1H-imidazol-3-ium tetrafluoroborate iodide (14)

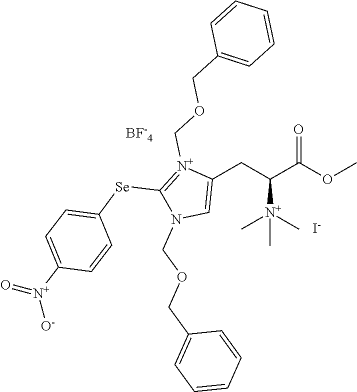 [SMILES: COC(=O)[C@H](Cc1cn(COCc2ccccc2)c([Se]c2ccc([N+](=O)[O-])cc2)[n+]1COCc1ccccc1)[N+](C)(C)C ; "BF-4" ; [I-]]

To a solution containing (S)-1,3-bis((benzyloxy)methyl)-4-(2-(dimethylamino)-3-methoxy-3-oxopropyl)-2-((4-nitrophenyl)selanyl)-1H-imidazol-3-ium tetrafluoroborate (13) (72.5 mg, 0.1 mmol) in 5 mL of THF was added iodomethane (18.7 μL, 0.3 mmol) and the reaction mixture was stirred for 24 h at room temperature. After that time, another 18.7 μL (0.3 mmol) of iodomethane was added and the reaction was allowed to stir for another 24 hours at room temperature. The reaction progress was monitored by HPLC-MS-PDA. The reaction mixture was evaporated to dryness affording the title compound (75%) which was subsequently used without further purification. HRMS (ESI-QTof) calcd. for C₃₂H₃₈N₄O₆Se (M+²) m/z=327.0978, found 327.0974. As previously mentioned, compound 14 being somewhat unstable, has the propensity of losing a Bom protecting group to provide compound 14A illustrated hereinbelow:

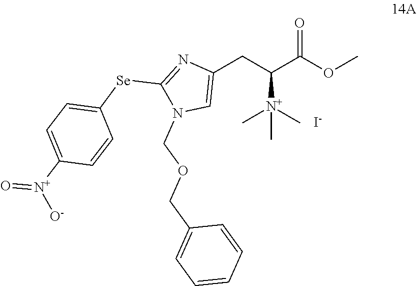 [SMILES: COC(=O)[C@H](Cc1cn(COCc2ccccc2)c([Se]c2ccc([N+](=O)[O-])cc2)n1)[N+](C)(C)C ; [I-]]

¹H NMR (300 MHz, CD₃CN) δ 8.04 (m, 2H), 7.44 (m, 2H), 7.38 (s, 1H), 7.20 (m, 5H), 5.47 (s, 2H), 4.49 (dd, J=10.7, 4.2 Hz, 1H_{β}), 4.41 (s, 2H), 3.68 (s, 3H), 3.32 (d, J=6.6 Hz, 1H), 3.29 (s, 9H), 3.27 (s, 1H p); HRMS (ESI-QTof) calcd. for C₂₄H₂₉N₄O₅Se (M⁺) m/z=533.1303, found 533.1300

Large scale: To a solution containing (S)-1,3-bis((benzyloxy)methyl)-4-(2-(dimethylamino)-3-methoxy-3-oxopropyl)-2-((4-nitrophenyl)selanyl)-1H-imidazol-3-ium tetrafluoroborate (13) (8.7 g 12.0 mmol) in 50 mL of THF was added iodomethane (2.2 mL, 63 mmol) and the reaction mixture was stirred for 24 h at room temperature. After that time, another 2.2 mL (63 mmol) of iodomethane was added and the reaction was allowed to stir for another 24 hours. The reaction progress was monitored by HPLC-MS-PDA. The reaction mixture was evaporated to dryness affording the title compound (75%) which was subsequently used without further purification.

### (S)-3-(2-selenoxo-2,3-dihydro-1H-imidazol-4-yl)-2-(trimethylammonio) propanoate (7) (as per Scheme 5)

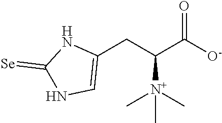

To a flask containing (S)-1,3-bis((benzyloxy)methyl)-4-(3-methoxy-3-oxo-2-(trimethylammonio)propyl)-2-((4-nitrophenyl)selanyl)-1H-imidazol-3-ium tetrafluoroborate iodide (14) (65.05 mg, 0.075 mmol) and 3-mercaptopropionic acid (0.65 mL, 7.5 mmol) was added 25 mL of concentrated HCl (36%) and the reaction mixture was allowed to stir under reflux for 26 h. Following solvent removal under vacuum, 20 mL of water were added, and the resulting mixture extracted with 3× (50 mL) methyl t-butyl ether (MTBE). The pH of the aqueous solution was adjusted to 6-7 with an aqueous NH₄OH solution (7%) under cooling and then evaporated to dryness. The purification of the residue by semi-preparative HPLC, followed by precipitation in absolute ethanol, gave the title compound (6.5 mg, 31%) as a yellow solid. ¹H NMR (300 MHz, D₂O) δ6.83 (s, 1H), 3.81 (dd, J=11.5, 4.2 Hz, 1H_{α}), 3.23 (m, 1H_{β}), 3.19 (s, 9H), 3.14 (m, 1H_{β}); ¹³C NMR (76 MHz, D₂O) δ170.02, 143.18, 126.29, 117.27, 77.11, 52.11, 22.87. HRMS (ESI-QTof) calcd. for C₉H₁₅N₃O₂Se (M+H⁺) m/z=278.0408, found 278.0406. [α]_{D}²⁵+110° (c 0.03, D₂O)

Large scale: To a flask containing (S)-1,3-bis((benzyloxy)methyl)-4-(3-methoxy-3-oxo-2-(trimethylammonio)propyl)-2-((4-nitrophenyl)selanyl)-1H-imidazol-3-ium tetrafluoroborate iodide (14) (7.8 g, 9 mmol) and 3-mercaptopropionic acid (78 mL, 900 mmol) was added 1 L of concentrated HCl (36%) and the reaction mixture was allowed to stir under reflux for 26 h. Following solvent removal under vacuum, 250 mL of water were added, and the resulting mixture extracted with 3× (500 mL) MTBE. The pH of the aqueous solution was adjusted to 6-7 with an aqueous solution of NH₄OH (7%) under cooling and then evaporated to dryness. The purification of the residue by flash chromatography, followed by precipitation in absolute ethanol, gave the title compound (520 mg, 21%) as a yellow solid.

All of the compounds and/or methods disclosed and claimed herein can be made and executed without undue experimentation in light of the present disclosure. While the compounds and methods of this disclosure have been described in terms of preferred embodiments, it will be apparent to those of skill in the art that variations may be applied to the compounds and/or methods and in the steps or in the sequence of steps of the methods described herein without departing from the concept, spirit, and scope of the disclosure. More specifically, it will be apparent that certain agents which are chemically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

1. Kryukov, G. V.; Castellano, S.; Novoselov, S. V.; Lobanov, A. V.; Zehtab, O.; Guigó, R.; et al., 2003. Characterization of mammalian selenoproteomes. Science 300, 1439-1443.
2. Labunskyy, V. M.; Hatfield, D. L.; Gladyshev, V. N. 2014. Selenoproteins: molecular pathways and physiological roles. Physiol. Rev. 94, 739-777.
3. Yamashita, Y.; Yabu, T.; Yamashita, M. 2010. Discovery of the strong antioxidant selenoneine in tuna and selenium redox metabolism. World J. Biol. Chem. 1, 144-150.
4. Klein M.; Ouerdane L.; Bueno M.; Pannier F. Identification in human urine and blood of a novel selenium metabolite, Se-methylselenoneine, a potential biomarker of metabolization in mammals of the naturally occurring selenoneine, by HPLC coupled to electrospray hybrid linear ion trap-orbital ion trap MS. Metallomics. 2011; 3(5):513-20.
5. Yamashita, M.; Yamashita, Y.; Ando, T.; Wakamiya, J.; Akiba, S. 2013a. Identification and determination of selenoneine, 2-selenyl-Nα, Nα, Nα-trimethyl-L-histidine, as the major organic selenium in blood cells in a fish-eating population on remote Japanese Islands. Biol. Trace Elem. Res. 156, 36-44.
6. Achouba A.; Dumas P.; Ouellet N.; Little M.; Lemire M.; Ayotte P. Selenoneine is a major selenium species in beluga skin and red blood cells of Inuit from Nunavik. Chemosphere. 2019; 229:549-558.
7. Little M.; Achouba A.; Dumas P.; Ouellet N.; Ayotte P.; Lemire M. Determinants of selenoneine concentration in red blood cells of Inuit from Nunavik (Northern Québec, Canada). Environ Int. 2019; 127:243-252.
8. Yamashita, M.; Yamashita, Y.; Suzuki, T.; Kani, Y.; Mizusawa, N.; Imamura, S., et al., 2013b. Selenoneine, a novel selenium-containing compound, mediates detoxification mechanisms against methylmercury accumulation and toxicity in zebrafish embryo. Mar. Biotechnol. N. Y. 15, 559-570.
9. Seko, T.; Imamura, S.; Ishihara, K. et al. Inhibition of angiotensin-converting enzyme by selenoneine. Fish Sci. 85, 731-736 (2019).
10. Pearson, R. G.; Sobel, H. R.; and Songstad, J. (1968) Nucleophilic reactivity constants toward methyl iodide and transdichlorodi(pyridine)platinum(II). J. Am. Chem. Soc. 90, 319-326. Halvorsen, A.; and Songstad, J. (1978) The reactivity of 2-bromo-1-phenylethanone (phenacyl bromide) toward nucleophilic species. J. Chem. Soc., Chem. Commun., 327-328.
11. Bachrach, S. M.; Demoin, D. W.; Luk, M.; Miller, J. V., Jr., Nucleophilic Attack at Selenium in Diselenides and Selenosulfides. A Computational Study, J. Phys. Chem. A, 2004, 108, 4040-4046.
12. Huber, R. E.; and Criddle, R. S. (1967) Comparison of the chemical properties of selenocysteine and selenocystine with their sulfur analog. Arch. Biochem. Biophys. 122, 164-173.
13. Besse D.; Budisa N.; Karnbrock W.; Minks C.; Musiol H. J.; Pegoraro S.; Siedler F.; Weyher E.; Moroder L. Chalcogen-analogs of amino acids. Their use in X-ray crystallographic and folding studies of peptides and proteins. Biol Chem. 1997; 378(3-4):211-8.
14. Lim D.; GrOndemann D.; Seebeck F. P. Total Synthesis and Functional Characterization of Selenoneine. Angew. Chem. Int. Ed. Engl. 2019; 58(42):15026-15030.
15. Hegedus, L. L.; McCabe, R. W. Catalyst Poisoning; Marcel Dekker: New York, NY, USA, 1984.
16. Miroslav Trampota. (2008). Process for the synthesis of 1-(+)-ergothioneine (US Patent Publication No. US 2009/0093642A1).
17. Rongnan Yi; Sen Liu; Hongxia Gao; Zhiwu Liang; Xinhua Xu; Ningbo Li. Iodine-promoted direct thiolation (selenylation) of imidazole with disulfides (diselenide): A convenient and metal-free protocol for the synthesis of 2-arylthio(seleno)imidazole, Tetrahedron, Volume 76, Issue 9, 2020.
18. Turrini, N. G.; Kroepfl, N.; Jensen, K. B.; Reiter, T. C.; Francesconi, K. A.; Schwerdtle, T.; Kroutil, W.; Kuehnelt, D. Biosynthesis and isolation of selenoneine from genetically modified fission yeast, Metallomics, 10 (2018) 1532-1538.
19. Marjanovic, B.; Simic, M. G.; Jovanovic, S. V. Heterocyclic thiols as antioxidants: Why ovothiol C is a better antioxidant than ergothioneine. Free Radic. Biol Med. 1995; 18(4):679-85.
20. Strober, W. Trypan Blue Exclusion Test of Cell Viability. Curr. Protoc. Immunol. 2015 Nov. 2; 111:A3.B.1-A3.B.3.
21. Antunes Dos Santos, A; Ferrer, B; Marques Gonçalves, F; Tsatsakis, A. M.; Renieri, E. A.; Skalny, A. V.; Farina, M.; Rocha, J. B. T.; Aschner, M. Oxidative Stress in Methylmercury-Induced Cell Toxicity. Toxics. 2018 Aug. 9; 6(3):47.
22. Lim, D.; GrOndemann, D; Seebeck, F. P. Total Synthesis and Functional Characterization of Selenoneine. Angewandte Chemie International Edition, 2019, 58(42), 15026-15030.
23. Nakamura, T.; Yoshida, K.; Yabuuchi, H.; Maeda, T.; Tamai, I. Functional Characterization of Ergothioneine Transport by Rat Organic Cation/Carnitine Transporter Octn1 (slc22a4). Biological and Pharmaceutical Bulletin, 2008, 31(8), 1580-1584.

1. A method of preparing a selenium compound of formula (I): 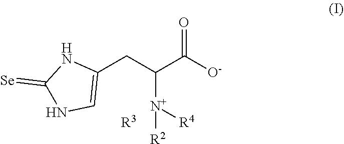
wherein R², R³ and R⁴ are each independently alkyl, the method comprising:
reacting a histidine ester under conditions sufficient to provide a dialkylated histidine ester; protecting the amino groups of the imidazole moiety of the dialkylated histidine ester and introducing a selone functional group at C2 of the imidazole moiety; deprotecting the amino groups of the imidazole moiety; protecting the imidazole N—H and selone functional groups; alkylating the tertiary amine and removing the protecting groups to provide the selenium compound of formula I.

2. A method of preparing a selenium containing compound of formula (I): 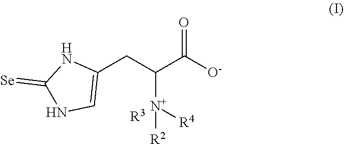
wherein R², R³ and R⁴ are each independently alkyl;
the method comprising reacting a compound of formula 2′: 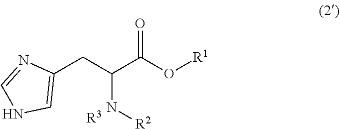
wherein R¹, R² and R³ are each independently alkyl, with an amine protecting agent followed by a selenation reaction to provide a compound of formula 3′: 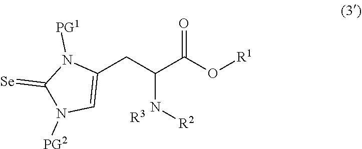
wherein R¹, R² and R³ are each independently alkyl and wherein PG¹ and PG² are amine protecting groups, and wherein PG¹ and PG² may be identical or different.

3. The method of claim 2, further comprising removing the amino protecting groups from the compound of formula 3′, to provide a compound of formula 4′: 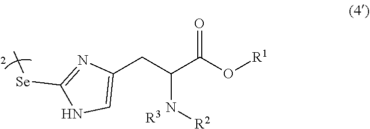
wherein R¹, R² and R³ are each independently alkyl.

4. The method of claim 3, further comprising reacting the compound of formula 4′ with a protecting agent to provide a compound of formula 5′: 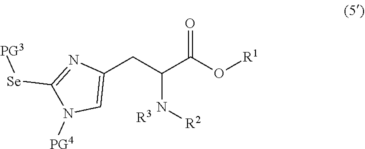
wherein R¹, R² and R³ are each independently alkyl and wherein PG³ and PG⁴ are an amine protecting group and a selenium protecting group respectively, wherein PG³ and PG⁴ may be identical or different.

5. The method of claim 4, further comprising quaternization of the tertiary amine and removing the protecting groups from the compound of formula 5′, to provide the compound of formula I.

6. The method of any one of claims 2 to 5, wherein the compound of formula 2′ is:

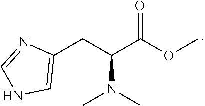

7. The method of any one of claims 2 to 6, wherein the compound of formula 3′ is:

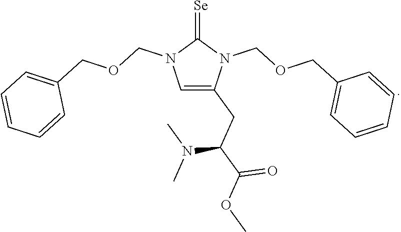 [SMILES: COC(=O)[C@H](Cc1cn(COCc2ccccc2)c(=[Se])n1COCc1ccccc1)N(C)C]

8. The method of any one of claims 2 to 7, wherein the compound of formula 4′ is:

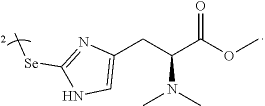

9. The method of any one of claims 2 to 8, wherein the compound of formula 5′ is:

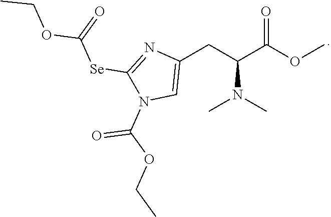 [SMILES: CCOC(=O)[Se]c1nc(C[C@@H](C(=O)OC)N(C)C)cn1C(=O)OCC]

10. The method of any one of claims 2 to 9, wherein the compound of formula I is:

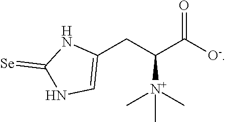

11. A method of preparing a selenium compound of formula (II) 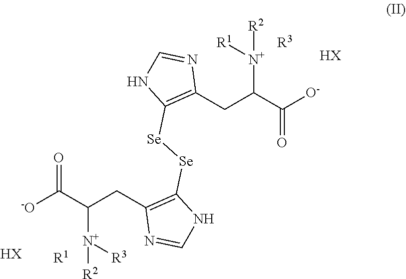
wherein R¹, R² and R³ are each independently alkyl and X is a halogen, the method comprising:
reacting a hercynine derivative under conditions sufficient for introducing a selenium at C5 of the imidazole moiety; and reacting the C5 seleno-substituted derivative to provide the selenium compound of formula II.

12. A method of preparing a selenium containing compound of formula (II): 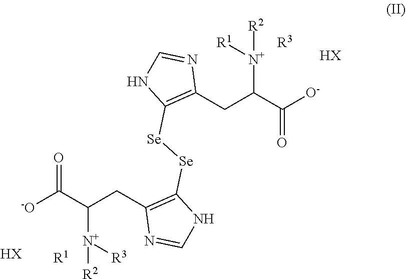
wherein R¹, R² and R³ are each independently alkyl and X is a halogen, the method comprising reacting a compound of formula 8′ 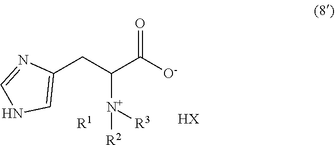
wherein R¹, R² and R³ are each independently alkyl and X is a halogen, with a selenation reagent to provide a compound of formula 9′ 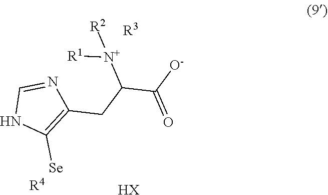
wherein R¹, R² and R³ are each independently alkyl; wherein R⁴ is a substituted alkyl group, wherein the substituent is a leaving group; and wherein X is a halogen.

13. The method of claim 12, further comprising subjecting the compound of formula 9′, to a deprotection reaction to provide the compound of formula II.

14. The method of claim 13, wherein the substitution reaction comprises a β-elimination reaction.

15. The method of any one of claims 12 to 14, wherein the compound of formula 8′ is:

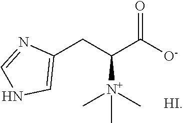

16. The method of any one of claims 12 to 15, wherein the compound of formula 9′ is:

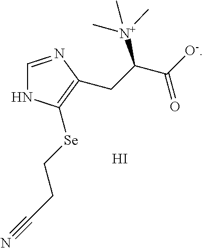

17. The method of any one of claims 12 to 16, wherein the compound of formula II is:

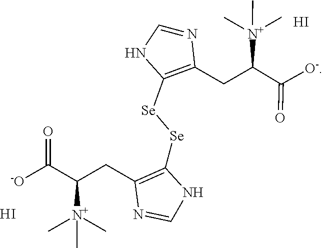

18. A method of preparing a selenium compound of formula (I): 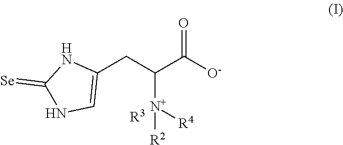
wherein R², R³ and R⁴ are each independently alkyl, the method comprising:
reacting a histidine ester under conditions sufficient to provide a dialkylated histidine ester; protecting the amino groups of the imidazole moiety of the dialkylated histidine ester and introducing a selone functional group at C2 of the imidazole moiety; protecting the selone functional group; alkylating the tertiary amine and removing the protecting groups to provide the selenium compound of formula I.

19. A method of preparing a selenium containing compound of formula (I): 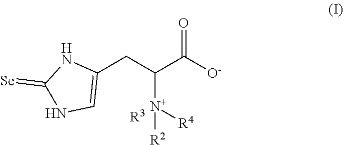
wherein R², R³ and R⁴ are each independently alkyl;
the method comprising reacting a compound of formula 2′: 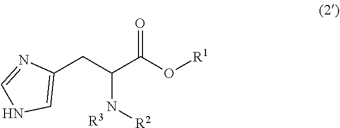
wherein R¹, R² and R³ are each independently alkyl, with an amine protecting agent followed by a selenation reaction to provide a compound of formula 3′: 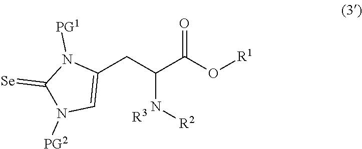
wherein R¹, R² and R³ are each independently alkyl and wherein PG¹ and PG² are amine protecting groups, and wherein PG¹ and PG² may be identical or different.

20. The method of claim 19, further comprising reacting the compound of formula 3′ with a protecting agent to provide a compound of formula 13′: 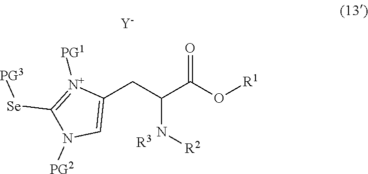
wherein R¹, R² and R³ are each independently alkyl, and wherein PG¹, PG² and PG³ are amine and selenium protecting groups respectively, wherein PG¹, PG² and PG³ may be identical or different.

21. The method of claim 20, further comprising quaternization of the tertiary amine of the compound of formula 13′ to provide a compound of formula 14′: 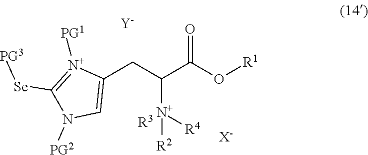
wherein R¹, R², R³ and R⁴ are each independently alkyl, and wherein PG¹, PG² and PG³ are amine and selenium protecting groups respectively, wherein PG¹, PG² and PG³ may be identical or different.

22. The method of claim 21, further comprising removing the protecting groups from the compound of formula 14′, to provide the compound of formula I.

23. The method of any one of claims 19 to 22, wherein the compound of formula 2′ is:

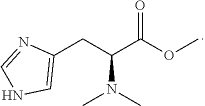

24. The method of any one of claims 19 to 23, wherein the compound of formula 3′ is:

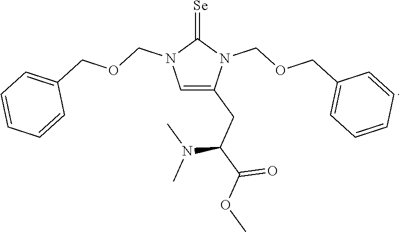 [SMILES: COC(=O)[C@H](Cc1cn(COCc2ccccc2)c(=[Se])n1COCc1ccccc1)N(C)C]

25. The method of any one of claims 19 to 24, wherein the compound of formula 13′ is:

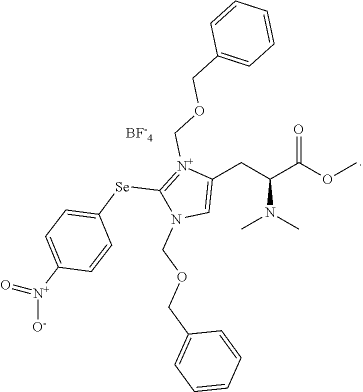 [SMILES: COC(=O)[C@H](Cc1cn(COCc2ccccc2)c([Se]c2ccc([N+](=O)[O-])cc2)[n+]1COCc1ccccc1)N(C)C ; "BF-4"]

26. The method of any one of claims 19 to 25, wherein the compound of formula 14′ is:

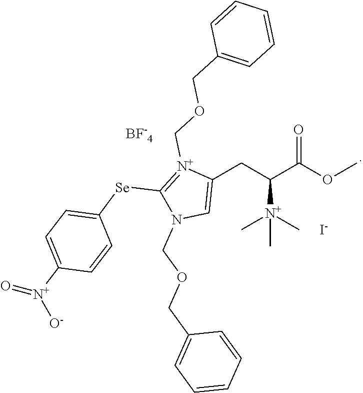 [SMILES: COC(=O)[C@H](Cc1cn(COCc2ccccc2)c([Se]c2ccc([N+](=O)[O-])cc2)[n+]1COCc1ccccc1)[N+](C)(C)C ; "BF-4" ; [I-]]

27. The method of any one of claims 19 to 26, wherein the compound of formula I is:

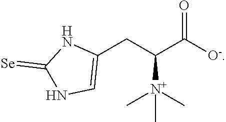

28. A method for improving an antioxidant effect that involves selenium in a subject, the method comprising administering a composition containing an effective amount of a compound of formula I: 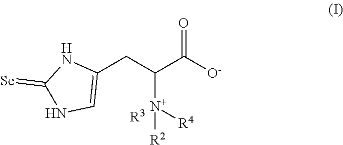
wherein R², R³ and R⁴ are each independently alkyl, and wherein the compound of formula I is obtained according to the method of any one of claims 1 to 10 or 19 to 28.

29. The method of claim 28, wherein the subject is a human or an animal.

30. The method of claim 28 or 29, wherein the composition is a drug, a functional food, a nutritional supplement, a food additive, an animal drug, a feed additive, or an antioxidant.

31. A method for inhibiting oxidation in a cell or tissue, the method comprising administering a composition containing an effective amount of a compound of formula I: 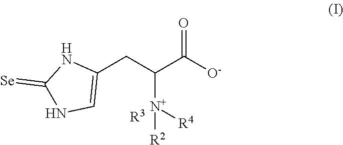
wherein R², R³ and R⁴ are each independently alkyl, and wherein the compound of formula I is obtained according to the method of any one of claims 1 to 10 or 19 to 28.

32. The method of claim 31, wherein the composition is effective for inhibiting or treating cytotoxic effects caused by a reactive oxygen species and/or methylmercury chloride (MeHgCl).

33. The method of claim 32, wherein the reactive oxygen species is a peroxide.

34. The method of claim 33, wherein the peroxide is t-butyl hydroperoxide (t-BuOOH).
